(19) United States
(12) Patent Application Publication
(10) Pub. No.: US 2002/0058834 A1
(43) Pub. Date: May 16, 2002

(54) Preparation of N-acyl amino carboxylic acids, amino carboxylic acids and their derivatives by metal-catalyzed carboxymethylation in the presence of a promoter

(76) Inventor: Thaddeus S. Franczyk II, Chesterfield, MO (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 09/871,869

(22) Filed: Jun. 1, 2001

Related U.S. Application Data
(60) Provisional application No. 60/208,692, filed on Jun. 1, 2000.

(51) Int. Cl. C07C 277/02

(52) U.S. Cl. 562/517

(57) ABSTRACT

A process for the preparation of amino carboxylic acids, N-acyl amino carboxylic acids, or derivatives thereof by carboxymethylation of an amide, amide precursor or amide source compound in the presence of a carboxymethylation catalyst precursor and a promoter is provided. A carboxymethylation reaction mixture is formed by introducing a promoter, an amide, amide precursor or amide source compound, carbon monoxide, hydrogen, an aldehyde or aldehyde source compound, and a carboxymethylation catalyst precursor into a carboxymethylation reaction zone. In a preferred embodiment, the promoter is a supported noble metal promoter. In another preferred embodiment, the amide compound and aldehyde are selected to yield an N-acyl amino carboxylic acid which is readily converted to N-phosphonomethyl)glycine, or a salt or ester thereof.

### BACKGROUND OF THE INVENTION

1. Field of the Invention

The present invention relates, in general, to the preparation of amino carboxylic acids, N-acyl amino carboxylic acids, and their derivatives, such as salts, amides, or esters, and, in a preferred embodiment, to the preparation of N-(phosphonomethyl)glycine, its salts, its amides, or its esters, wherein the method of preparation comprises carboxymethylation of an amide, amide precursor or an amide source compound in the presence of a carboxymethylation catalyst precursor and a promoter.

2. Description of Related Art

Amino carboxylic acids, N-acyl amino carboxylic acids, and their derivatives are useful in various applications. For example, N-acyl sarcosinates are useful as adjuvants for pesticide formulations as disclosed in U.S. Pat. No. 5,985,798 and references cited therein. Glycine, for example, is widely used as an additive in processed meat, beverages, and in other processed food stuffs. It is also used widely as a raw material for pharmaceuticals, agricultural chemicals, and pesticides. N-(phosphonomethyl)glycine, also known by its common name glyphosate, is a highly effective and commercially important herbicide useful for combating the presence of a wide variety of unwanted vegetation, including agricultural weeds. N-acetyl glycine is a useful chemical intermediate for the preparation of amino acids such as phenylalanine. N-acyl amino acids in which the acyl group is derived from a long chain fatty acid are useful as chelating agents, surfactants, detergents, and lubricating agents. Convenient and economical methods of preparing glyphosate and other amino carboxylic acids are, therefore, of great importance.

Preparation of amino carboxylic acids, N-acyl amino carboxylic acids, and their derivatives by the carboxymethylation of amides has been previously disclosed. The synthesis of N-acetyl α-amino acids using aldehydes such as formaldehyde was first reported by Wakamatsu et al. in Chemical Communications, 1540 (1970) which describes the preparation of monocarboxymethylated amides such as N-acetyl glycine in 46% yield.

J-J Lin et al. disclosed in U.S. Pat. No. 4,918,222 an improved method for preparing N-acetyl glycine by a cobalt-catalyzed carboxymethylation of acetamide in the presence of a non-metallic promoter organic solvent. Stern et al. in Synthetic Communications, 12, 1111-13 (1982) disclose a cobalt-catalyzed preparation of N-acetyl iminodiacetic acid using carbon monoxide, hydrogen, formaldehyde, and acetamide.

Stern et al. in PCT application publication number WO 9835930 disclose an improved process for preparing 2-aminocarboxylic acids by carboxymethylation, and a number of methods for converting the acids to glyphosate. The PCT application further discloses the use of a strong mineral acid as a co-catalyst.

Hirai et al. disclose the preparation of certain N-acyl α-amino acids in Tetrahedron Letters, 23(24), 2491-4 (1982) by the cobalt-catalyzed carboxymethylation of acylamides using allylic alcohols wherein a homogeneous complexed rhodium, iron, or palladium co-catalyst is said to isomerize the allylic alcohol to an aldehyde. The disclosed overall procedure of Hirai et al. is not useful in preparing N-acyl glycines or N-acyl iminodiacetic acids.

Beller et al. in Angewandte Chemie, International Edition in English, 36, 1494-96 (1997) report the preparation of N-acyl amino acids by carboxymethylation of amides catalyzed by complexed palladium in the absence of a cobalt carbonylation catalyst and as an alternative to cobalt catalyzed carboxymethylation of amides. Belier et al. further disclose the use of a mineral acid, sulfuric acid, as a co-catalyst or non-metallic promoter to accelerate the reaction rate. The Beller et al. process requires the presence of a source of halide ions which can be corrosive limiting its suitability for industrial processes.

Franz, et al. in Glyphosate: A Unique Global Herbicide (ACS Monograph 189, 1997) at p. 233-257 identify a number of processes by which glyphosate can be prepared. According to one of these, iminodiacetic acid disodium salt (DSIDA) is treated with formaldehyde and phosphorous acid or phosphorous trichloride to produce N-(phosphonomethyl) iminodiacetic acid and sodium chloride. One of the carboxymethyl groups on N-(phosphonomethyl) iminodiacetic acid is then oxidatively cleaved in the presence of a carbon catalyst to produce glyphosate acid. A significant drawback of this method is that it produces as a side product three equivalents of sodium chloride per equivalent of glyphosate. Sodium chloride waste streams of this nature are difficult to recycle because typically after precipitation the salt contains significant quantities of entrapped organic matter. Such entrapped organic matter prevents the sodium chloride from being used for many purposes, for example in foods or feed. Further recrystallization of the sodium chloride adds cost which makes recycle economically unattractive. Alternative methods of disposing sodium chloride without detriment to the environment are expensive and difficult. Franz et al. (at pages 242-243) also describe a method in which N-isopropyl glycine is phosphonomethylated to produce N-isopropyl-N-(phosphonomethyl)glycine which is then converted to glyphosate. In this method, the N-isopropyl-N-(phosphonomethyl)glycine is heated to 300° C. with 50% sodium hydroxide and then treated with hydrochloric acid to produce glyphosate. The severe and costly conditions necessary to cleave the N-isopropyl group represent a significant disadvantage of that method. In addition, this method also produces a significant sodium chloride waste stream.

In U.S. Pat. No. 4,400,330, Wong discloses a method for the preparation of glyphosate in which 2,5-diketopiperazine is reacted with paraformaldehyde and a phosphorous trihalide in a carboxylic acid solvent to produce N,N-bis(phosphonomethyl)-2,5-diketopiperazine which is then saponified to form a glyphosate sodium salt. The Wong method is limited by the fact that diketopiperazine is a relatively expensive starting material. Furthermore, the conversion of glyphosate sodium salt to the acid form or to other salts produces an undesired sodium chloride waste stream.

The present invention provides a promoter to accelerate a metal-catalyzed carboxymethylation reaction without the need to use prior art non-metallic promoters such as a special solvent or strong mineral acids. The present invention further provides a promoter to carry out a metal-catalyzed carboxymethylation reaction at lower pressures, with reduced amounts of carboxymethylation catalyst, and with improved yields of product. Specifically, N-acyl amino acids such as N-acetyl glycine, N-acyl sarcosinates and N-acetyl iminodiacetic acid can be prepared with a promoter in combination with a cobalt catalyst precursor at lower pressures, with reduced amounts of carboxymethylation catalyst, and with improved yields without special reaction-promoting solvents, strong mineral acids, or corrosive halides.

### SUMMARY OF THE INVENTION

Among the objects of the present invention, therefore, is the provision of a well-defined, low-cost process for the production of N-acyl amino carboxylic acids, amino carboxylic acids or their derivatives in general, N-acetyl iminodiacetic acid in particular, and iminodiacetic acid in a preferred embodiment. In another preferred embodiment, the amino carboxylic acid or its derivative is converted to N-(phosphonomethyl)glycine in a process in which sodium chloride is not generated as a by-product.

Briefly, therefore, the present invention is directed to a process for the preparation of an amino carboxylic acid or a derivative thereof by carboxymethylation of an amide, an amide precursor or an amide source compound. The process comprises introducing a carboxymethylation catalyst precursor, a promoter for conversion of the catalyst precursor to an active catalyst species, an aldehyde or an aldehyde source compound, carbon monoxide, hydrogen and the amide, amide precursor or amide source compound into a carboxymethylation reaction zone to form a carboxymethylation reaction mixture. The carboxymethylation reaction mixture is then heated under pressure to produce a carboxymethylation product mixture containing an amino carboxylic acid product, a catalyst precursor reaction product and the promoter.

In one embodiment, the amide, amide precursor or amide source compound comprises a compound having the formula:

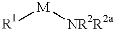

wherein;

M is selected from the group consisting of C(O), S(O), S(O)₂, P(O)OH, and P(O)R¹⁸;

R¹ is selected from the group consisting of hydrogen, hydrocarbyl, substituted hydrocarbyl, —NR³R⁴, —OR⁵, and —SR⁶;

R² and R²′ are independently selected from the group consisting of hydrogen, hydrocarbyl, and substituted hydrocarbyl;

R³ and R⁴ are independently selected from the group consisting of hydrogen, hydrocarbyl, and substituted hydrocarbyl;

R⁵ and R⁶ are independently selected from the group consisting of hydrogen, hydrocarbyl, substituted hydrocarbyl, and a salt-forming cation;

M and NR²R^{2a} are taken together to form a C≡N provided that R¹ is selected from the group consisting of hydrocarbyl and substituted hydrocarbyl; and

R¹⁸ is NR²R^{2a};

provided, however, that at least one of R² and R^{2a} is a group which undergoes carbonylation under the conditions of said carboxymethylation reaction; or R¹ is —NR³R⁴ and at least one of R³ and R⁴ is a group which undergoes carbonylation under the conditions of said carboxymethylation reaction.

In another embodiment, the amide compound is a carbamoyl compound and the amino carboxylic acid product in the carboxymethylation product mixture comprises an N-acyl amino carboxylic acid or a derivative thereof, wherein the carbamoyl compound has the formula:

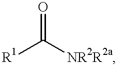

wherein;

R¹ is selected from the group consisting of hydrogen, hydrocarbyl, substituted hydrocarbyl, NR³R⁴, OR⁵, and SR⁶;

R² and R^{2a} are independently selected from the group consisting of hydrogen, hydrocarbyl, and substituted hydrocarbyl;

R³ and R⁴ are independently selected from the group consisting of hydrogen, hydrocarbyl, and substituted hydrocarbyl; and

R⁵ and R⁶ are independently selected from the group consisting of hydrogen, hydrocarbyl, substituted hydrocarbyl, and a salt-forming cation;

provided, however, that at least one of R² and R^{2a} is a group which undergoes carbonylation under the conditions of the carboxymethylation reaction; or R¹ is —NR³R⁴ and at least one of R³ and R⁴ is a group which undergoes carbonylation under the conditions of carboxymethylation reaction.

In a preferred embodiment, the amide compound and aldehyde are selected to yield an N-acyl amino carboxylic acid which is convertible in one or more steps to N-(phosphonomethyl)glycine, or a salt or ester thereof having the following structure I:

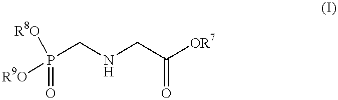

wherein R⁷, R⁸, and R⁹ independently are hydrogen, hydrocarbyl, substituted hydrocarbyl, or an agronomically acceptable cation.

The present invention is also directed to a process for the preparation of an amino carboxylic acid or a derivative thereof by carboxymethylation of an amide compound. The process comprises introducing a carboxymethylation catalyst precursor, a promoter for conversion of the catalyst precursor to an active catalyst species, carbon monoxide, hydrogen, an aldehyde or an aldehyde source compound, and said amide compound into a carboxymethylation reaction zone to form a carboxymethylation reaction mixture. The carboxymethylation reaction mixture is then heated under pressure to produce a carboxymethylation product mixture containing an N-acyl amino carboxylic acid product, a catalyst precursor reaction product, and the promoter. The promoter is recovered from the carboxymethylation product mixture, and the catalyst precursor reaction product is recovered from the carboxymethylation product mixture and regenerated in the presence of the amide compound. The amide compound comprises a carbamoyl compound having the formula:

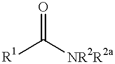

wherein;

R¹ is selected from the group consisting of hydrocarbyl and substituted hydrocarbyl; and

R² and R^{2a} are independently selected from the group consisting of hydrogen, hydrocarbyl, and substituted hydrocarbyl;

provided, however, that at least one of R² and R^{2a} is a group which undergoes carbonylation under the conditions of the carboxymethylation reaction.

Further, the present invention is directed to a process for the preparation of an amino carboxylic acid or a derivative thereof by carboxymethylation of an amide compound comprising a sulfonamide or sulfinamide compound having the formula;

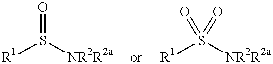

wherein;

R¹ is selected from the group consisting of hydrocarbyl, substituted hydrocarbyl, and NR³R⁴;

R² and R^{2a} are independently selected from the group consisting of hydrogen, hydrocarbyl, and substituted hydrocarbyl; and

R³ and R⁴ are independently selected from the group consisting of hydrogen, hydrocarbyl, and substituted hydrocarbyl;

provided, however, that at least one of R² and R^{2a} is a group which undergoes carbonylation under the conditions of the carboxymethylation reaction; or R¹ is —NR³R⁴ and at least one of R³ and R⁴ is a group which undergoes carbonylation under the conditions of the carboxymethylation reaction. The process comprises introducing a carboxymethylation catalyst precursor, a promoter for conversion of the catalyst precursor to an active catalyst species, carbon monoxide, hydrogen, an aldehyde or an aldehyde source compound, and the amide compound into a carboxymethylation reaction zone to form a carboxymethylation reaction mixture. The carboxymethylation reaction mixture is then heated under pressure to produce a carboxymethylation product mixture containing an amino carboxylic acid product, a catalyst precursor reaction product, and the promoter.

Still further, the present invention is directed to a process for the preparation of N-(phosphonomethyl)glycine or a salt or ester thereof. The process comprises preparing an N-acyl iminodiacetic acid by carboxymethylating an acylamide in a carboxymethylation reaction mixture formed by introducing the acylamide, water, formaldehyde, carbon monoxide, hydrogen, a supported noble metal promoter, and a carboxymethylation catalyst precursor comprising cobalt into a carboxymethylation reaction zone. The N-acyl iminodiacetic acid is then deacylated to convert the N-acyl iminodiacetic acid to N-(phosphonomethyl)glycine or a salt or ester thereof.

Still further, the present invention is directed to a process for the preparation of N-(phosphonomethyl)glycine, a salt of N-(phosphonomethyl)glycine, or an ester of N-(phosphonomethyl)glycine. The process comprises preparing an N-acyl amino carboxylic acid product by carboxymethylating a carbamoyl compound in a carboxymethylation reaction mixture formed by introducing the carbamoyl compound, formaldehyde, carbon monoxide, hydrogen, a carboxymethylation catalyst precursor comprising cobalt and a supported metallic promoter into a carboxymethylation reaction zone. The N-acyl amino carboxylic acid product is then converted to N-(phosphonomethyl)glycine, a salt of N-(phosphonomethyl)glycine, or an ester of N-(phosphonomethyl)glycine by deacylating the N-acyl amino carboxylic acid product to generate a carboxylic acid and an amino acid. The resulting carboxylic acid is then reacted with an amine to generate the carbamoyl compound or a compound from which the carbamoyl compound may be derived.

The present invention is still further directed to a process for the preparation of N-(phosphonomethyl)glycine, a salt of N-(phosphonomethyl)glycine, or an ester of N-(phosphonomethyl)glycine. The process comprises preparing N-acetyl iminodiacetic acid by carboxymethylating acetamide in a carboxymethylation reaction mixture formed by introducing acetamide, acetic acid, water, formaldehyde, carbon monoxide, hydrogen, a carboxymethylation catalyst precursor comprising cobalt and a supported noble metal promoter into a carboxymethylation reaction zone. The N-acetyl iminodiacetic acid is then converted to N-(phosphonomethyl)glycine, a salt of N-(phosphonomethyl)glycine, or an ester of N-(phosphonomethyl)glycine by deacylating the N-acetyl iminodiacetic acid.

Still further, the present invention is directed to a process for the preparation of N-(phosphonomethyl)glycine, a salt of N-(phosphonomethyl)glycine, or an ester of N-(phosphonomethyl)glycine from a carbamoyl compound having the formula:

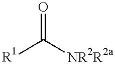

wherein;

R¹ is —NR³R⁴;

R² and R^{2a} are independently hydrogen, hydrocarbyl, or substituted hydrocarbyl; and

R³ and R⁴ are independently hydrogen, hydrocarbyl, or substituted hydrocarbyl;

provided, however, that at least one of R², R^{2a}, R³ and R⁴ is hydrogen, hydroxymethyl, amidomethyl, or another substituent which undergoes carbonylation under the conditions of the carboxymethylation reaction .. The process comprises introducing the carbamoyl compound, a carboxymethylation catalyst precursor, a supported noble metal promoter, formaldehyde, and carbon monoxide to a carboxymethylation reaction zone to form a carboxymethylation reaction mixture. The carboxymethylation reaction mixture is then heated under pressure to produce a carboxymethylation product mixture containing an N-acyl amino carboxylic acid product and a catalyst precursor reaction product. The N-acyl amino carboxylic acid product is then converted to N-(phosphonomethyl)glycine, a salt of N-(phosphonomethyl)glycine, or an ester of N-(phosphonomethyl)glycine.

Still further, the present invention is directed to a process for the preparation of N-(phosphonomethyl)glycine, a salt of N-(phosphonomethyl)glycine, or an ester of N-(phosphonomethyl)glycine from a carbamoyl compound having the formula:

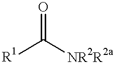

wherein;

R¹ is alkyl;

R² is hydrocarbyl or substituted hydrocarbyl; and

R^{2a} is hydrogen, hydroxymethyl, or another substituent which is carbonylated under the carboxymethylation reaction conditions. The process comprises introducing the carbamoyl compound, a carboxymethylation catalyst precursor, a supported noble metal promoter, formaldehyde, and carbon monoxide into a carboxymethylation reaction zone to form a carboxymethylation reaction mixture. The carboxymethylation reaction mixture is then reacted to produce a carboxymethylation product mixture containing an N-acyl-N-alkyl amino carboxylic acid product and a catalyst precursor reaction product. The N-acyl-N-alkyl amino carboxylic acid reaction product is then converted to an N-alkyl-N-(phosphonomethyl)glycine compound, and the N-alkyl-N-(phosphonomethyl)glycine compound is oxidatively dealkylated with a noble metal catalyst in the presence of oxygen to form N-(phosphonomethyl)glycine or an ester or salt thereof.

The present invention is additionally directed to the certain key starting materials used and intermediates prepared in the process of the present invention. Further scope of the applicability of the present invention will become apparent from the detailed description provided below. It should be understood, however, that the following detailed description and examples, while indicating preferred embodiments of the invention, are given by way of illustration only since various changes and modifications within the spirit and scope of the invention will become apparent to those skilled in the art from this detailed description.

### DETAILED DESCRIPTION OF THE INVENTION

The process of the present invention is broadly directed to the carboxymethylation of amide, amide precursor, or amide source compounds in which a strong acid co-catalyst, a halide, or anhydrous conditions are not required and in which a promoter and a carboxymethylation catalyst precursor are present. A preferred embodiment of this process is schematically depicted in Reaction Scheme 1 in which hydridocobalttetracarbonyl is identified for convenience of discussion as the carboxymethylation catalyst precursor, the amide is a carbamoyl compound, and the promoter is a supported metallic promoter.

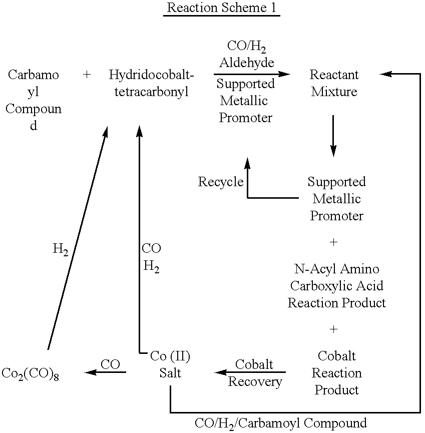

As depicted, a carbamoyl compound is introduced into a carboxymethylation reaction zone with hydridocobalttetracarbonyl in the presence of a supported metallic promoter, carbon monoxide, hydrogen, and an aldehyde (or source of aldehyde) to produce a carboxymethylation product mixture comprising an N-acyl amino carboxylic acid reaction product and a cobalt catalyst reaction product. The N-acyl amino carboxylic acid reaction product may then be deacylated, for example, by hydrolysis, or otherwise further processed as may be desired.

The hydridocobalttetracarbonyl for use as a carboxymethylation catalyst precursor process may be obtained in any one of several ways. For example, in one embodiment of the present invention, the hydridocobalttetracarbonyl is generated in situ in the carboxymethylation reaction mixture by contacting the carbamoyl compound and dicobaltoctacarbonyl (or other catalyst precursor) in the presence of hydrogen, carbon monoxide, a supported metallic promoter and an aldehyde. As depicted in Reaction Scheme 1, the dicobaltoctacarbonyl may be obtained by recycle and regeneration of a cobalt(II) salt which is recovered from a prior carboxymethylation reaction. Recovery of a cobalt(II) salt for conversion to dicobaltoctacarbonyl is described in Weisenfeld, Ind. Eng. Chem. Res., Vol.31, No.2, p.636-638 (1992). In a second embodiment of the present invention, the cobalt(II) salt is regenerated and treated with carbon monoxide and hydrogen by conventional techniques to produce hydridocobalttetracarbonyl. In a third embodiment of the present invention, the cobalt(II) salt is converted to hydridocobalttetracarbonyl using carbon monoxide and hydrogen in the presence of the carbamoyl compound. When aldehyde is then introduced to the hydridocobalttetracarbonyl and carbamoyl compound mixture, the resulting reaction mixture yields the N-acyl amino carboxylic acid product of the carboxymethylation reaction.

A. Carboxymethylation Reaction

The carboxymethylation reaction of the present invention comprises the reaction of an amide, an amide precursor, or an amide source compound with an aldehyde or an aldehyde source compound in the presence of a carboxymethylation catalyst precursor, a promoter, carbon monoxide and hydrogen.

In general, the amide compound is a sulfonamide, phosphonamide, or a carbamoyl compound such as an N-acyl amide, a urea, or a carbamate corresponding to the formula II:

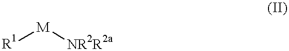

wherein M is selected from the group consisting of C(O), S(O), S(O)₂, P(O)OH, and P(O)R¹⁸; R¹ is selected from the group consisting of hydrogen, hydrocarbyl, substituted hydrocarbyl, —NR³R⁴, —OR⁵, or —SR⁶; R² and R^{2a} are independently selected from the group consisting of hydrogen, hydrocarbyl, or substituted hydrocarbyl; R³ and R⁴ are independently selected from the group consisting of hydrogen, hydrocarbyl, or substituted hydrocarbyl; R⁵ and R⁶ are independently selected from the group consisting of hydrogen, hydrocarbyl, substituted hydrocarbyl, or a salt-forming cation;

M and NR²R^{2a} are taken together to form a C≡N, provided that R¹ is selected from the group consisting of hydrocarbyl and substituted hydrocarbyl; and

R¹⁸ is NR²R^{2a};

provided, however, that at least one of R² and R^{2a} is a group which undergoes carbonylation under the conditions of the carboxymethylation reaction; or R¹ is —NR³R⁴ and at least one of R³ and R⁴ is a group which undergoes carbonylation under the conditions of carboxymethylation reaction.

Preferably the amide is a carbamoyl compound having the structure IIa:

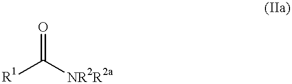

wherein R¹, R² and R^{2a} are as previously defined.

In one embodiment of the present invention, R¹ is hydrocarbyl or substituted hydrocarbyl, typically a C₁ to about C₃₀ hydrocarbyl or substituted hydrocarbyl. In this embodiment, R¹ is preferably C₁ to about C₂₀ hydrocarbyl or substituted hydrocarbyl, more preferably C₁ to about C₁₀ hydrocarbyl or substituted hydrocarbyl, most preferably C₁ to about C₆hydrocarbyl or substituted hydrocarbyl, and even more preferably C₁hydrocarbyl or substituted hydrocarbyl.

In another embodiment of the present invention, R¹ is —NR³R⁴. In this embodiment, R³ and R⁴ are independently hydrogen, hydrocarbyl or substituted hydrocarbyl. In general, if either of R³ and R⁴ is hydrocarbyl, it is a C₁ to about C₂₀ hydrocarbyl, preferably C₁ to about C₁₀ hydrocarbyl, more preferably C₁ to about C₆ hydrocarbyl, and still more preferably methyl or isopropyl. If R³ or R⁴ is substituted hydrocarbyl, typically it is C₁ to about C₂₀ substituted hydrocarbyl, preferably C₁ to about C₁₀ substituted hydrocarbyl, more preferably C₁ to about C₆ substituted hydrocarbyl, and still more preferably it is phosphonomethyl (—CH₂PO₃H₂), hydroxymethyl (—CH₂OH), amidomethyl (—CH₂N(R¹²)C(O)R¹¹), carboxymethyl (—CH₂CO₂H), or an ester or salt of carboxymethyl or phosphonomethyl. If R² and R^{2a} are each hydrocarbyl or substituted hydrocarbyl, it is required that at least one of R³ and R⁴ is a group which undergoes carbonylation under the conditions of the carboxymethylation reaction. In general, preferred amidomethyl substituents correspond to the radical:

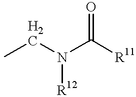

wherein R¹¹ and R¹² are independently hydrogen, hydrocarbyl, substituted hydrocarbyl, hydroxymethyl, carboxymethyl, phosphonomethyl, or an ester or salt of carboxymethyl or phosphphonomethyl.

Preferably, at least one of R² and R^{2a} is hydrogen, hydroxymethyl, or amidomethyl. More preferably, at least one of R² and R^{2a} is hydrogen. If, however, R² or R^{2a} is hydrocarbyl, it is typically a C₁ to about C₂₀ hydrocarbyl, preferably C₁ to about C₁₀ hydrocarbyl, more preferably C₁ to about C₆ hydrocarbyl, and still more preferably methyl or isopropyl. If R² or R^{2a} is substituted hydrocarbyl, typically it is C₁ to about C₂₀ substituted hydrocarbyl, preferably C₁ to about C₁₀ substituted hydrocarbyl, and more preferably C₁ to about C₆ substituted hydrocarbyl. The substituted hydrocarbyl can be, for example, phosphonomethyl, hydroxymethyl, amidomethyl, carboxymethyl, an ester or salt of carboxymethyl or phosphonomethyl, or (N′-alkylamido)methyl, preferably phosphonomethyl, carboxymethyl, amidomethyl or an ester or salt of carboxymethyl or phosphphonomethyl. It is possible for R² and R^{2a} to be non-identical. For example, R² can be hydrocarbyl and R^{2a} can be substituted hydrocarbyl. In another embodiment, R² can be an alkyl such as methyl while R^{2a} can be, for example, hydroxymethyl or an (N′-alkylamido)methyl group such as (N′-methylamido)methyl.

The carboxymethylation catalyst precursor may be any composition which is known to be useful in catalyzing carboxymethylation reactions and which generally contains a metal from Group VIII of the Periodic Table (CAS version). These compositions are referred to as “carboxymethylation catalyst precursors” herein since the precise form of the catalyst participating in the reaction is not known with certainty. In any event, the carboxymethylation catalyst precursor preferably comprises cobalt. Without being bound by a particular theory, it is believed that a reduced form of cobalt acts as the active catalyst species in the carboxymethylation reactions of the present invention. Still more preferably the carboxymethylation catalyst precursor is selected from a group consisting of cobalt metal, cobalt oxide, organic and inorganic salts of cobalt (for example, halides such as cobalt chloride and cobalt bromide), aromatic and aliphatic carboxylates of cobalt (such as cobalt acetate, cobalt propionate, cobalt octanoate, cobalt stearate, cobalt benzoate and cobalt naphthenate), and complex cobalt compounds containing one or more ligands such as carbonyls, nitrites and phosphines. The preferred cobalt-containing carboxymethylation catalyst precursors are dicobaltoctacarbonyl (Co₂(CO)₈), hydridocobalttetracarbonyl (HCo(CO)₄), cobalt tetracarbonyl anion ([Co(CO)₄]⁻¹) or a cobalt(II) salt.

The reactants can be introduced to the carboxymethylation reaction zone in a number of ways. For example, the amide, amide precursor or amide source compound is generally introduced to the reaction zone simultaneously with the carboxymethylation catalyst precursor or as a pre-mix of amide compound and catalyst precursor. Alternatively, the promoter may also be introduced along with the amide compound and the catalyst precursor. Further, the amide compound and the carboxymethylation catalyst precursor may be introduced to the reaction zone in the presence of the aldehyde (or an aldehyde source compound which may contain water or an aldehyde source compound which may be a part of the amide) and/or carbon monoxide. The promoter may be added with the aldehyde (or an aldehyde source compound which may contain water) and/or carbon monoxide or added separately, prior or subsequently to the aldehyde or carbon monoxide.

When the amide compound is a carbamoyl compound such as acetamide, the reaction mixture may be formed by introducing the amide, aldehyde source compound, carbon monoxide, hydrogen, a promoter, and carboxymethylation catalyst precursor to the carboxymethylation reaction zone without premixing the amide and the carboxymethylation catalyst precursor. However, it is important to note that, when the amide compound is a carbamoyl compound of the urea or a substituted urea type such as bis-phosphonomethylurea, the carbamoyl compound and carboxymethylation catalyst precursor are preferably introduced to the reaction zone in the essential absence of water or aldehyde source compounds which contain water. The resulting mixture of carbamoyl compound and carboxymethylation catalyst precursor is then contacted with the aldehyde source compound, carbon monoxide, and hydrogen in the carboxymethylation reaction zone. The promoter may be present when the carbamoyl compound and carboxymethylation catalyst precursor is combined or added along with the aldehyde source compound, carbon monoxide, and hydrogen.

The promoter is a material that accelerates the carboxymethylation reaction while being unable to significantly and directly function as a carboxymethylation catalyst. It has been found that the presence of a promoter in the carboxymethylation reaction zone facilitates the conversion of the carboxymethylation catalyst precursor to an active catalyst species and typically requires less carboxymethylation catalyst precursor to be charged to the carboxymethylation reaction zone than a similar carboxymethylation reaction conducted in the absence of a promoter.

Without being held to a particular theory, it is generally believed that the promoter may be any material which is capable of directly facilitating the cleavage of a hydrogen-hydrogen bond within the carboxymethylation reaction zone, thus enhancing the ability of molecular hydrogen present in the reaction zone to reduce the carboxymethylation catalyst precursor. By enhancing the ability of molecular hydrogen to act as a reducing agent for the carboxymethylation catalyst precursor, the promoter may also allow for the reaction to be conducted at a lower partial pressure of hydrogen, and thus a lower overall operating pressure, than a similar carboxymethylation reaction conducted in the absence of a promoter. Further, it has been found that the presence of a promoter in the carboxymethylation reaction mixture helps to stabilize the active catalyst species in the reaction mixture. Therefore, the promoter may independently allow for the reaction to be conducted at a lower partial pressure of stabilizing carbon monoxide, and thus a lower overall operating pressure, than a similar carboxymethylation reaction conducted in the absence of a promoter.

Preferably, the promoter is a metallic promoter. The metal may be in any form which accelerates the carboxymethylation reaction directly or in a form which is converted to an accelerating form during the conditions of the process. The metal that is a part of a metallic promoter is preferably deposited on the surface of a support; or it may be complexed by one or more ligands, be a free metal, or present in the form of a cation or anion part of a salt. Preferably, the metal of the metallic promoter is a noble metal. Suitable noble metals for use as a metallic promoter include platinum (Pt), palladium (Pd), rhenium (Re), rhodium (Rh), or ruthenium (Ru). Preferred noble metals are platinum and palladium with palladium being most preferred. The noble metal can be deposited on an inert support to give a supported noble metal promoter. Supported noble metal promoters are preferred due to their high reactivity and ease of recovery of the metallic promoter after the reaction. The supported noble metal promoters are more preferably platinum and palladium with supported palladium the most preferred. Because supported palladium is presently most preferred, much of the following discussion will be directed to the use of supported palladium. However, it should be understood that the following discussion is generally applicable to the other metallic promoters and particularly supported noble metal promoters and combinations thereof.

Preferably, the promoter comprises a noble metal on a support; with a supported palladium promoter being particularly preferred. The noble metal may be supported on a variety of materials. For example, suitable supports include, but are not limited to, carbon, alumina (Al₂O₃), silica (SiO₂), titania (TiO₂), zirconia (ZrO₂), siloxane, barium sulfate (BaSO₄) or mixtures thereof. Preferably, the support is a form of carbon. Supported noble metals useful as metallic promoters are commercially available from various sources such as Aldrich Chemical Co., Inc., Milwaukee, Wis.; Engelhard Corp, Iselin, N.J.; and Degussa Corp., Ridgefield Park, N.J. and include, for example, 5% palladium on activated carbon, Aldrich Catalog No. 20,568-0; 10% palladium on activated carbon, Aldrich Catalog No. 20,569-9; 5% palladium on alumina powder, Aldrich Catalog No. 20,571-0; palladium on barium sulfate (reduced), Aldrich Catalog No. 27,799-1; and 5% Platinum on activated carbon, Aldrich Catalog No. 20,593-1).

The concentration of the noble metal on the surface of the support may vary within wide limits, preferably in the range of from about 0.5 to about 15 or 20% by weight of the support ([mass of noble metal÷total mass of the supported noble metal promoter]×100%), and more preferably from about 1 to about 10% by weight of the support. At concentrations greater than about 20% by weight, layers and clumps of noble metal tend to form at the surface of the support. Thus, there are fewer surface noble metal atoms per total amount of noble metal used. This tends to reduce catalyst activity and may be an uneconomical use of the noble metal.

The molar ratio of the noble metal component of the metallic promoter to the amide, amide precursor, or amide source compound in the carboxymethylation reaction mixture can vary widely. Generally, one of ordinary skill in the art will recognize that the effectiveness of the promoter depends on many factors such as the surface area, type of support, type of metal, the physical form of the metal and the like, and, further, that it is advantageous to minimize amount of noble metal. Thus, the mole ratio of the noble metal component to the amide, amide precursor, or amide source compound typically ranges from about 0.0001 to about 0.05, preferably from about 0.001 to about 0.01, and more preferably from about 0.002 to about 0.005.

The carboxymethylation reaction is typically conducted under pressure. Generally, the reaction pressure can vary widely, for example, with pressures ranging from about 100 psi to about 4000 psi or more (about 700 kPa to about 28,000 kPa or more). The pressure is generated principally by the addition and maintenance of carbon monoxide and hydrogen in the carboxymethylation reaction mixture. However, one or more diluent gases also may be included in the carboxymethylation reaction zone. Preferably, the carboxymethylation reaction is conducted at a pressure ranging from about 500 to about 3500 psi (about 3,500 kPa to about 24,500 kPa), and more preferably from about 600 to about 3250 psi (about 4,200 kPa to about 22,750 kPa).

In the carboxymethylation reaction, hydrogen and, optionally, other diluent gases such as nitrogen or helium, are introduced to the carboxymethylation reaction zone with the carbon monoxide to maintain a partial pressure of hydrogen in the vapor phase of the carboxymethylation reaction mixture. Typically, the ratio of carbon monoxide to hydrogen in the carboxymethylation reaction mixture, on a partial pressure basis, is at least about 1:1, preferably from about 70:30 to about 99:1, and more preferably from about 85:15 to about 97:3.

In general, the carboxymethylation reaction can be run at any temperature at which the reactants and equipment can be conveniently handled and the carboxymethylation reaction proceeds. Typically, the reaction temperature ranges from about 50° C. to about 170° C., preferably from about 65° C to about 140° C., more preferably from about 80° C. to about 130° C., and still more preferably from about 95° C. to about 115° C.

The molar ratio of carboxymethylation catalyst precursor metal atoms to amide, amide precursor or amide source compound in the carboxymethylation reaction zone can vary widely. Typically, it is desired to maintain a low ratio of catalyst precursor to amide compound in the reaction zone. For example, the molar ratio of carboxymethylation catalyst precursor to amide, amide precursor or amide source compound may be varied over the range of from about 0.001 to about 1, preferably from about 0.01 to about 0.5, and more preferably from about 0.02 to about 0.1.

Aldehydes useful in the process of the present invention may be present in a molecular form, in a partially or fully polymeric form, in an aqueous solution, as an acetal, as an N-hydroxy hydrocarbyl adduct of the amide, as an N-hydroxy substituted hydrocarbyl adduct of the amide, or as a result of hydroformylation of an olefin during the carboxymethylation reaction. A broad range of aldehydes can be used. For example, the aldehyde may contain more than one formyl group and, in addition to the oxygen atom(s) of the formyl group(s), the aldehyde may contain other oxygen atoms or other heteroatoms, such as in furfurylacetaldehyde, 4-acetoxyphenylacetaldehyde, phenylacetaldehyde, and 3-methylthiopropionaldehyde. Typically, suitable aldehydes are of the general formula R—CHO wherein R is hydrogen, hydrocarbyl, or substituted hydrocarbyl. In general, R contains up to 20 carbon atoms, more suitably up to 10 carbon atoms. Examples of such aldehydes are phenylacetaldehyde, formylcyclohexane, and 4-methylbenzaldehyde. Preferably, R is hydrogen, a linear or branched alkyl group containing up to 6 carbon atoms, or an aralkyl group where the aryl contains 6 to 12 carbon atoms and the alkyl contains up to 6 carbon atoms. More preferably, the aldehyde is formaldehyde, acetaldehyde, 3-methylthiopropionaldehyde or isobutyraldehyde, and in a particularly preferred embodiment, the aldehyde is formaldehyde with the source of the formaldehyde being formalin.

In alternative embodiments, the carboxymethylation reaction of the present invention may further include one or more acid co-catalysts or the reaction may be conducted in the presence of a solvent. For some amide compounds such as acetamide, amide precursors such as nitrites, and amide source compounds such as acetamide equivalents, the acid co-catalyst is preferably an organic acid, such as a carboxylic acid, having a pKa greater than about 3 or a mineral acid having a pKa of less than 3. The organic acid co-catalyst can be, for example, formic acid, acetic acid, or propionic acid, preferably formic acid or acetic acid, and most preferably acetic acid. The mineral acid co-catalyst can be, for example, sulfuric acid, hydrochloric acid, hydrobromic acid, a strong organic acid such as methanesulfonic acid, trifluoroacetic acid, toluenesulfonic acid, and the like. In general, when the amide compound is an acyl amide, a preferred organic acid co-catalyst is the carboxylic acid which corresponds to the acyl amide (i.e., the carboxylic acid which is a part of the amide).

The carboxymethylation reaction can be further conducted in the presence of a solvent which is chemically and physically compatible with the reaction mixture. Preferably, the solvent is a weaker base than the amide compound. The solvent can be, for example, an ether, a ketone, an ester, a nitrile, a carboxylic acid, a tertiary amide such as dimethylformamide or N-methyl pyrrolidone, or a mixture thereof. Preferably, the solvent is an ether, a ketone, or a nitrile; more particularly dimethoxyethane (DME), tetrahydrofuran (THF), acetone, 2-butanone, acetonitrile, acetic acid, or t-butyl methyl ether.

In a preferred embodiment, the carboxymethylation reaction is carried out in the presence of water. In this embodiment, the molar ratio of water to the amide compound in the carboxymethylation reaction mixture is generally less than about 10, preferably between about 2 and about 5, and more preferably between about 3 and about 4.

Payload, which is measured as the mass of amide, amide precursor, or amide source compound divided by the mass of reaction solvent, may be varied over a wide range. One skilled in the art will recognize that useful ranges of payload will depend in part on the physical state of the amide compound starting material under the reaction conditions employed and its compatibility with solvents used, if any. The payload typically varies through the range of from about 0.001 grams of amide compound per gram of solvent (gc/gs) in the reaction mixture to about 1 gc/gs. Preferably, payload is at least about 0.01 gc/gs, more preferably at least about 0.1 gc/gs, still more preferably from about 0.12 gc/gs to about 0.35 gc/gs, and in a particularly preferred embodiment, from about 0.15 gc/gs to about 0.3 gc/gs.

The reaction can be carried out in a batch mode or in a continuous mode. When run in a continuous mode, the residence time in the reaction zone can vary widely depending on the specific reactants and conditions employed, with typical residence times varying over the range of about 1 minute to about 500 minutes, preferably about 10 minutes to about 250 minutes, and more preferably about 30 minutes to about 100 minutes. When run in a batch mode, reaction time typically varies over the range of about 10 seconds to about 12 hours, preferably about 2 minutes to about 6 hours, and more preferably about 10 minutes to about 3 hours.

B. Promoter Recovery

After the carboxymethylation reaction, the promoter is preferably recovered for possible reuse as a promoter in subsequent carboxymethylation reactions. The nature of the recovery step will vary depending on the type and form of promoter used. When the promoter is a metallic promoter, any recovery method of the metallic promoter which is compatible with the carboxymethylation reaction mixture and products can be used provided the promoting ability of the metallic promoter is retained. Suitable general procedures to recover the metallic promoter, when the metallic promoter is in the preferred form of a supported noble metal promoter, include, but are not limited to, filtration of the carboxymethylation product mixture, centrifugation of the product mixture, containerization of the metallic promoter inside a closed, porous containment vessel inside the reactor, and circulation of the carboxymethylation reaction mixture through the metallic promoter contained in a filter system attached to the reactor.

A suitable specific procedure for removal of a supported noble metal promoter from a carboxymethylation reaction mixture is to pass the carboxymethylation product mixture through a filter so as to retain the metallic promoter (i.e., noble metal on support). The filter can then be backwashed with solvents of the carboxymethylation reaction to return the metallic promoter to the carboxymethylation reaction zone for use in a subsequent carboxymethylation reaction.

C. Carboxymethylation Catalyst Recovery

After the carboxymethylation reaction and following removal of the promoter from the carboxymethylation product mixture, the catalyst precursor reaction product is preferably recovered and the carboxymethylation catalyst precursor regenerated for reuse in a subsequent carboxymethylation reaction. The nature of the recovery step will vary depending on the catalyst precursor and generally, any recovery method of the catalyst precursor which is compatible with the carboxymethylation reaction mixture and carboxymethylation products may be used.

Weisenfeld (U.S. Pat. No. 4,954,466) reported a method of recovering cobalt catalyst values from carboxymethylation product mixtures wherein a cobalt-N-acetyl iminodiacetic acid complex was dissolved in an aqueous solution with a strong acid and then extracted with a hydrocarbon solvent containing a trialkylamine to transfer the cobalt from the aqueous solution into the hydrocarbon solvent. The cobalt was then stripped from the hydrocarbon solvent with water and precipitated with a strong base.

Another method for the recovery of cobalt catalyst values from carboxymethylation product mixtures is described in European Patent Application Publication No. EP 0 779 102 A1. In that method, cobalt is recovered from carboxymethylation product mixtures such as those yielding N-acyl sarcosine by treatment of the finished reaction mixture with aqueous hydrogen peroxide or aqueous hydrogen peroxide and sulfuric acid, thereby converting the cobalt catalyst to water-soluble cobalt(II) salts. The aqueous phase containing the water-soluble cobalt(II) salts is then separated from the nonaqueous phase. Excess hydrogen peroxide is next removed from the aqueous phase, for example, by beating. An alkali metal hydroxide is then added to the aqueous phase causing the precipitation of cobalt(II) hydroxide. The cobalt(II) hydroxide is collected and washed in preparation for regeneration to cobalt catalyst.

Alternatively, and in accordance with one aspect of the present invention, cobalt from a carboxymethylation product mixture is oxidized to a soluble cobalt(II) species. The oxidation step is carried out by exposing the carboxymethylation product mixture to a molecular oxygen-containing gas for a suitable length of time. The exposure to oxygen may be achieved by any convenient means, for example, by bubbling an oxygen-containing gas through the product mixture or by maintaining an atmosphere of an oxygen-containing gas over the reaction mixture. The progress of the reaction can be monitored by color changes in which the final oxidized system is a deep red or red-purple color which undergoes no further changes. Alternatively, the progress of the reaction can be monitored by infrared spectroscopy or by cyclic voltametry.

The concentration of molecular oxygen in the oxygen-containing gas used in the cobalt recovery step of the present invention can vary depending on the reaction conditions. The concentration of oxygen typically ranges from about 0.1% by weight to about 100% by weight, with higher concentrations of oxygen in the oxygen-containing gas typically causing faster oxidation reaction rates. However, relatively low concentrations of oxygen in the oxygen-containing gas are favored when volatile organic solvents are present in the reaction mixture because of inherent safety risks. Preferably, the concentration of oxygen in the oxygen-containing gas is from about 5 wt. % to about 80 wt. %, more preferably from about 10 wt. % to about 30 wt. %.

The oxygen-containing gas can also contain a diluent gas. Preferably the diluent gas is inert under the reaction conditions. Typical diluent gases include nitrogen, helium, neon, and argon, with nitrogen being preferred. Air can conveniently be used as the oxygen-containing gas. The oxidation may be carried out under subatmospheric pressure, atmospheric pressure, or superatmospheric pressure. Preferably, the oxidation is conducted at pressures ranging from about 10 psi to about 100 psi (about 70 to about 700 kPa), and more preferably from about 30 psi to about 60 psi (about 200 to about 400 kPa).

The oxidized cobalt(II) species may be converted in situ into an insoluble cobalt(II) salt complex with the amino carboxylic acid reaction product of the carboxymethylation product mixture by allowing the product mixture to stand for a suitable length of time. For example, it is convenient to allow the product mixture to stand overnight to achieve the precipitation of the insoluble cobalt(II) salt complex. As the insoluble cobalt(II) salt complex forms, it precipitates out of solution.

Formation and precipitation of the insoluble cobalt(II) salt complex can be accelerated by raising the temperature of the system. The temperature of the carboxymethylation product mixture during the oxidation step and during the complex-formation step of the present invention typically ranges from about room temperature to about 150° C., preferably from about 60° C. to about 110° C., and more preferably from about 70° C. to about 100° C.

Further, the formation and precipitation of the oxidized cobalt(II) salts also may be facilitated by the presence of a composition such as an organic acid (for example, formic, acetic, oxalic, or propionic acid) which may be present in the carboxymethylation reaction. Alternatively, the composition may be separately introduced to the carboxymethylation product mixture after the carboxymethylation reaction. The insoluble cobalt(II) salt complex can be separated from the product mixture by any convenient means, for example, by filtration or centrifugation, and subsequently used to regenerate fresh cobalt catalyst precursor for use in additional carboxymethylation reactions. The oxidation of the catalyst precursor product in the carboxymethylation product mixture to the cobalt(II) species and conversion of the cobalt(II) species to an insoluble cobalt(II) salt complex can optionally be performed as two discrete steps or combined into a single step in which oxidation and salt formation are carried out in a nearly simultaneous fashion.

As a further alternative, the formation and precipitation of the oxidized cobalt(II) salts may also be accelerated by the addition of a solvent. Typical solvents include dimethylether (“DME”), acetone, or any solvent suitable in the carboxymethylation reaction. In general, the amount of excess solvent is at least about 50% of the volume of the reaction mass, more preferably from about 75% to about 150% of the reaction mass, and most preferably from about 90% to about 110% of the reaction mass.

Instead of introducing molecular oxygen to the carboxymethylation product mixture, the cobalt(II) species may be formed under anaerobic conditions. In this procedure, the product mixture is simply heated after the carboxymethylation reaction pressure has been reduced. Typically, the product mixture is heated to reflux or distilled at a temperature of from about 60° C. to about 100° C. to effect the oxidation cobalt catalyst reaction product to a cobalt(II) salt. The cobalt(II) salt may then be precipitated or extracted from the carboxymethylation product mixture using any of the procedures described above. In addition, the formation and precipitation of the oxidized cobalt(II) salts may also be accelerated by the presence of an organic acid, a mineral acid or by the addition of a solvent as described above in the case where molecular oxygen is introduced to the carboxymethylation product mixture.

D. Carboxymethylation Catalyst Regeneration

Several methods for regenerating a cobalt catalyst have been reported in the literature which may be used in accordance with the present invention to regenerate carboxymethylation catalyst precursors comprising cobalt. For example, in U.S. Pat. No. 4,954,466, Weisenfeld suggests converting a cobalt(II) precipitate to dicobaltoctacarbonyl by reacting the precipitate with carbon monoxide and hydrogen at a temperature of 150° C. to 180° C. and a pressure of 1500 to 6000 psi (10,345 to 41,380 kPa).

European Patent Application Publication No. EP 0 779 102 A1 describes regenerating a carboxymethylation cobalt catalyst by recovering cobalt hydroxide from a carboxymethylation reaction introduced to the melt of an N-acyl amino acid derivative such as an N-acyl sarcosine. The mixture is then added to a polar aprotic solvent and reacted with carbon monoxide or a mixture of carbon monoxide and hydrogen to form a carboxymethylation catalytic mixture.

U.S. Pat. No. 5,321,168 describes a further method for regenerating a carboxymethylation cobalt catalyst. A solution of cobalt salts recovered from a carboxymethylation reaction are converted to carboxymethylation catalyst precursors in the presence of noble metals such as palladium, platinum or gold which may be unsupported or supported on silica, alumina, zeolite, or activated carbon. Alternatively, supports such as ion exchange resins containing amino groups, silica, alumina, zeolite, or activated carbon alone may suffice for converting cobalt salts recovered from a carboxymethylation reaction to carboxymethylation catalyst precursors. The formation of carboxymethylation catalyst precursors is then accomplished with a mixture of carbon monoxide and hydrogen at indicated temperatures, pressures, and time periods.

Surprisingly, it has been discovered that the rate of regeneration of the cobalt(II) salt can be dramatically increased if the regeneration is conducted in the presence of an amide compound of the present invention along with carbon monoxide and hydrogen. For example, when the amide compound is an N-acyl amide, productivity is significantly increased by regenerating the cobalt(II) salt in the presence of the N-acyl amide.

In accordance with the present invention, therefore, when the amide compound is an N-acyl amide, the cobalt(II) salt can be regenerated in the presence of the N-acyl amide, an aldehyde, the N-acyl amide and the aldehyde, or neither the N-acyl amide or the aldehyde. When the amide compound is urea (or another amide compound which is a less competent base than N-acyl amides), however, the cobalt(II) salt is preferably regenerated in the presence of the urea or other amide compound and in the essential absence of water or aldehyde source compounds which contain water. If the active catalyst mixture is regenerated in the absence of the amide compound, therefore, it is further advantageous to add the amide compound to the reaction mixture before the addition of an aldehyde source compound. For example, when the amide compound is a urea structure IIa wherein R¹ is —NR³R⁴, it is advantageous to treat the cobalt(II) salt with carbon monoxide, hydrogen, and urea before an aldehyde source compound is added to the reaction mixture.

During catalyst precursor regeneration, the reaction pressure generally ranges from about 200 psi to about 4,000 psi (about 1,400 to about 28,000 kPa), preferably from about 800 psi to about 3,700 psi (about 5,600 to about 26,000 kPa), and more preferably from about 1,500 psi to about 3,500 psi (about 10,500 kPa to about 24,000 kPa). In general, the ratio of carbon monoxide to hydrogen, on a partial pressure basis, during the catalyst precursor regeneration ranges from about 99:1 to about 1:99, preferably from about 30:70 to about 90:10, and more preferably from about 50:50 to about 75:25. The progress of the regeneration reaction can be followed by monitoring the uptake of gas, for example, by monitoring head pressure.

During the catalyst precursor regeneration, it is often advantageous to heat the reaction mixture. Typically, reaction mixture temperatures range from about 70° C. to about 170° C., preferably from about 90° C. to about 150° C., and more preferably from about 100° C. to about 140° C. Reaction times for the regeneration step can vary from about 1 minute to about 5 hours, preferably from about 5 minutes to about 2 hours, and more preferably from about 10 minutes to about 1 hour.

Alternatively, the regeneration step can be performed in the presence of an organic acid co-catalyst, if an organic acid co-catalyst is used in the carboxymethylation reaction. The regenerated active catalyst precursor complex can, if desired, be used in a carboxymethylation reaction directly after regeneration. The anionic portion of the cobalt(II) salt is not critical to the regeneration step. For example, the cobalt(II) species can be in the form of a salt of the conjugate base of the carboxymethylation reaction product from which the cobalt(II) species was recovered. Alternatively, the cobalt(II) species can be in any other convenient form such as cobalt acetate tetrahydrate, cobalt stearate, cobalt acetonate, or cobalt oxalate.

E. Deacylation

In many embodiments of the present invention, wherein the amino carboxylic acid product comprises an N-acyl amino carboxylic acid product, it is desirable to deacylate the N-acyl amino carboxylic acid product. Generally, such deacylation can be achieved by hydrolysis or by the formation of a diketopiperazine species.

In general, the N-acyl amino carboxylic acid reaction product is hydrolyzed in the presence of a hydrolysis catalyst, for example, an acid or a base, preferably a mineral acid. Suitable mineral acids useful for this purpose include hydrochloric acid, sulfuric acid, nitric acid, phosphoric acid, or phosphorous acid. Alternatively, the N-acyl reaction product may be hydrolyzed in the absence of a mineral acid or base merely by heating the N-acyl amino carboxylic acid reaction product in the presence of water to form the corresponding amino acid.

Instead of being hydrolyzed, the N-acyl amino carboxylic acid reaction product can be deacylated and cyclized in a single step, as depicted in Reaction Scheme 2, to form a 2,5-diketopiperazine which is useful, for example, as an intermediate in the preparation of N-(phosphonomethyl)glycine:

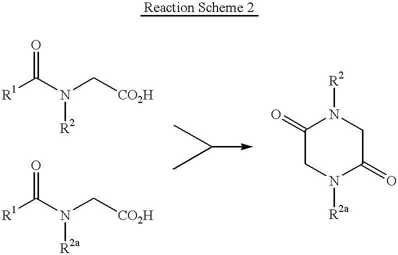

wherein R² and R^{2a} are independently hydrogen, alkyl, or carboxymethyl or the salts or esters thereof. Examples of such reactions include the preparation of 1,4-di(carboxymethyl)-2,5-diketopiperazine (XVII) from N-acetyl iminodiacetic acid (XVI) (see Reaction Scheme 7), the preparation of 2,5-diketopiperazine (XXX) from N-acetyl glycine (XVII) (see Reaction Scheme 9a), and the preparation of 1,4-dimethyl-2,5-diketopiperazine (XXXI) from N-acetyl-N-methyl glycine (XX) (See Reaction Schemes 11 and 15).

Typically, the reaction temperature for deacylating and cyclizing the N-acyl amino carboxylic acid reaction product ranges from about 100° C. to about 250° C., preferably EO from about 150° C. to about 220° C. and more preferably from about 185° C. to about 200° C. The reaction is relatively rapid, with reaction times typically ranging from about 1 minute to about 10 hours, preferably from about 5 minutes to about 5 hours, and more preferably from about 10 minutes to about 3 hours.

Typically, the reaction takes place in the presence of water. The amount of added water, measured as a percent of the starting material, can generally range up to about 85 wt. %, and preferably is from about 5 wt. % to about 70 wt. %, more preferably from about 9 wt. % to about 20 wt. %. If desired, a catalyst such as an organic acid can be added to the deacylation and cyclization reaction mixture. Preferably, such a catalyst comprises a C₁ to about C₃ carboxylic acid, and most preferably, the catalyst is acetic acid. Solvents can optionally be present in the reaction mixture. For example, ethers, ketones, or nitrites can be added.

The formation of a 2,5-diketopiperazine from the N-acyl amino acid reaction product by cyclization is advantageous for a number of reasons. As a general rule, 2,5-diketopiperazines are less soluble in many solvents and in water than is the corresponding amino carboxylic acid product. As a result, the diketopiperazine can be more readily precipitated from the product mixture, separated, and handled. Furthermore, since the deacylation-cyclization reaction does not require strong mineral acids, it is less corrosive to process equipment than a hydrolysis reaction in which strong mineral acids are employed.

The deacylation-cyclization and hydrolysis reactions of N-acyl amino carboxylic acid reaction products can occur simultaneously, resulting in a mixture of deacylation and hydrolysis products. This mixture of products can be subsequently used as produced, i.e., without separation and purification, or it can be separated into its component products. The ratio of hydrolysis and deacylation-cyclization products achieved in the final reaction mixture depends on the conditions selected for the reaction. For example, in Reaction Scheme 3, N-acetyl iminodiacetic acid (XVI) is heated in water to form iminodiacetic acid (XIV), 1,4-di(carboxymethyl)-2,5-diketopiperazine (XVII) or mixtures thereof. The ratio of XIV to XVII can be controlled as a function of the various conditions under which the reaction is performed as disclosed in PCT application publication number WO 9835930.

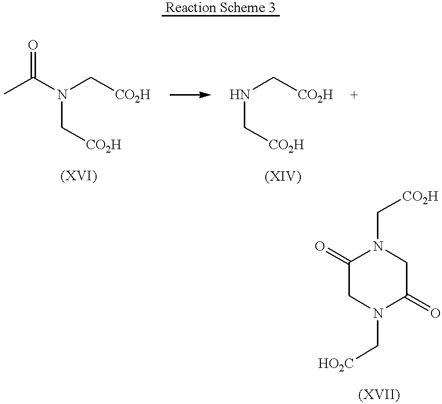

In general, a wide variety of N-acyl amino carboxylic acids prepared in the present process can be hydrolyzed or deacylated-cyclized using the conditions described herein. Examples of such N-acyl amino carboxylic acids and the products of the reactions as described herein are set forth in Table 1.

F. Phosphonomethylation

In certain embodiments of the present invention, it is preferred that the N-acyl amino carboxylic acid reaction product, or the hydrolysis or deacylation-cyclization product therefrom, be phosphonomethylated. Phosphonomethylation reactions of amines and of amino acids have been reported. For example, Moedritzer, et al. (J. Org. Chem. 1966, 31, 1603-1607) reported the reaction of primary and secondary amino acids with phosphorous acid and formaldehyde to form, respectively, di- and mono-phosphonomethylated amino acids. Moedritzer also reported (U.S. Pat. No.3,288,846) the reaction of iminodiacetic acid (XIV) with phosphorous acid and formaldehyde to prepare N-(phosphonomethyl) iminodiacetic acid (XV). Miller et al. (U.S. Pat. No. 4,657,705) disclose a process in which substituted ureas, amides and carbamates are phosphonomethylated to produce an N-substituted aminomethylphosphonic acid which can be converted to glyphosate. In the disclosed process, the urea, amide or carbamate is (1) mixed with an aqueous acidic medium comprising phosphorous acid and an acid selected from among sulfuric, hydrochloric and hydrobromic acids and (2) heated to a temperature between about 70° C. and about 120° C. Phosphonomethylation reactions can also be carried out using phosphorous trichloride instead of phosphorous acid as described in, for example, U.S. Pat. No. 4,400,330.

Typically, the N-acyl amino carboxylic acid product to be phosphonomethylated is treated with a source of phosphorous acid and a source of formaldehyde. Another mineral acid such as sulfuric acid or hydrochloric acid is preferably added. Reaction temperatures generally range from about 80° C. to about 150° C., preferably from about 100° C. to about 140° C., more preferably from about 120° C. to about 140° C. Reaction times generally range from about 10 minutes to about 5 hours, preferably from about 20 minutes to about 3 hours, more preferably from about 30 minutes to about 2 hours.

TABLE 1
Hydrolysis or Deacylation-Cyclization Product Examples

| N-acyl amino carboxylic | Hydrolysis | Deacylation-Cyclization |
|---|---|---|
| acid | Product | Product |
| N,N,N′,N′-tetra | Iminodiacetic | None |
| (carboxymethyl)urea (XIII) | acid (XIV) | |
| N-acetyl iminodiacetic | Iminodiacetic | 1,4-di(carboxymethyl)-2,5- |
| acid (XVI) | acid (XIV) | diketopiperazine (XVII) |
| N-acetyl-N- | N- | 1,4-di(phosphonomethyl)- |
| (phosphonomethyl) glycine | (phosphono- | 2,5-diketopiperazine |
| (XIX) | methyl) | |
| | glycine (I) | |
| N,N′-di(carboxymethyl)- | Sarcosine | None |
| N,N′-dimethyl urea (XXII) | (XXIII) | |
| N,N′-di(carboxymethyl)- | N- | None |
| N,N′-di- | (phosphono- | |
| (phosphonomethyl) urea | methyl) | |
| (XXIV) | glycine(I) | |
| N-acetyl-N-methyl glycine | N-methyl | 1,4-dimethyl-2,5- |
| (XX) | glycine (XXIII) | diketopiperazine (XXXI) |
| N-acetyl glycine (XVIII) | Glycine | 2,5-diketopiperazine |
| | | (XXX) |

Any source of phosphorous acid or phosphorous acid equivalent can be used in the phosphonomethylation reaction. For example, phosphorous acid, phosphorous trichloride, phosphorous tribromide, phosphorous acid esters, chlorophosphonic acid and esters of chlorophosphonic acid can be used. Phosphorous acid and phosphorous trichloride are preferred. Formaldehyde can be derived from any source, for example, paraformaldehyde or formalin.

In one embodiment of the present invention, the phosphonomethylation reaction results in the replacement of the N-acyl substituent of the N-acyl amino carboxylic acid product with an N-phosphonomethyl group to produce an N-(phosphonomethyl)amino acid. This reaction is shown generically in Reaction Scheme 4 wherein R¹ and R² are as previously defined. Examples of this type of reaction include the conversion of N-acyl sarcosine to N-methyl-N-(phosphonomethyl)glycine, conversion of N-acyl iminodiacetic acid to N-(phosphonomethyl) iminodiacetic acid, and the conversion of N-acyl glycine to glyphosate.

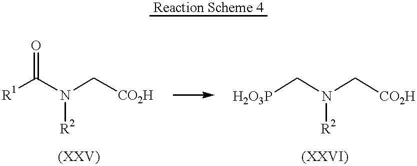

In another embodiment of the present invention, a 2,5-diketopiperazine is phosphonomethylated with phosphorous trichloride, phosphorous acid, or a source of phosphorous acid in the presence of a formaldehyde source compound to form N-substituted-N-(phosphonomethyl)glycine as shown in Reaction Scheme 4a:

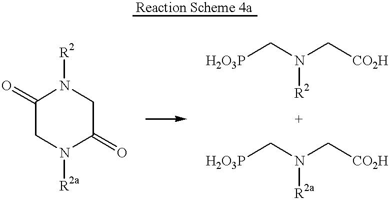

wherein R²and R^{2a} are independently hydrogen, alkyl, or carboxymethyl or the salts or esters thereof.

In a further embodiment of the present invention, an N-acyl glycine is phosphonomethylated to form N-(phosphonomethyl)glycine (I). For example, the reaction of N-acetyl glycine (XVIII), phosphorous acid or phosphorous trichloride, and a source of formaldehyde produces N-(phosphonomethyl)glycine (I) (See Reaction Scheme 9).

In a still further aspect of the present invention, N-acyl-N-alkyl glycine compounds can be phosphonomethylated to produce N-alkyl-N-(Phosphonomethyl)glycine compounds. For example, N-acetyl-N-methyl glycine (XX) can be reacted with a source of formaldehyde and with phosphorous acid or phosphorous trichloride to produce N-methyl-N-(phosphonomethyl)glycine (XXI) (See Reaction Schemes 12 and 16).

G. Oxidative Dealkylation

In another embodiment of the present invention, the N-acyl amino acid carboxymethylation reaction product is converted to N-alkyl-N-(phosphonomethyl)glycine (“N-substituted glyphosate”), which is oxidatively dealkylated to generate N-(phosphonomethyl)glycine, for example, as disclosed in PCT application publication number WO 98/35930. Preferably, oxidation is carried out by contacting the N-substituted glyphosate with water and feeding the resulting mixture into a reactor along with an oxygen-containing gas or a liquid containing dissolved oxygen. In the presence of a noble metal catalyst, the N-substituted glyphosate reactant is oxidatively converted into glyphosate and various byproducts:

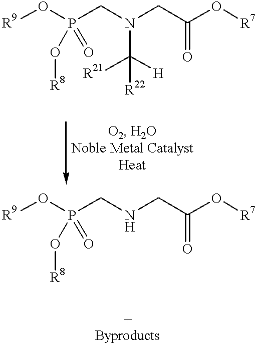

wherein R⁷, R⁸, and R⁹ are as previously defined, and R²¹ and R²² are independently hydrogen, halogen, —PO₃H₂, —SO₃H, —NO₂, hydrocarbyl or unsubstituted hydrocarbyl other than —CO₂H.

In a preferred embodiment, the catalyst subsequently is separated by filtration, and the glyphosate then is isolated, for example, by precipitation by evaporation of a portion of the water and cooling.

H. Preparation of Glyphosate

In a preferred embodiment of the present invention, the N-acyl amino carboxylic acid product of the carboxymethylation reaction is converted to glyphosate or one of its salts or esters having the structure I:

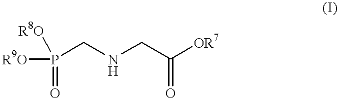

wherein R⁷, R⁸, and R⁹ independently comprise hydrogen, hydrocarbyl, substituted hydrocarbyl, or an agronomically acceptable cation. When R⁷, R⁸, and R⁹ of structure I are each hydrogen, the compound of structure I is glyphosate. Preferably, in the carboxymethylation reaction to produce the N-acyl amino carboxylic acid product for conversion to glyphosate, formaldehyde (or a formaldehyde source compound) is selected as the aldehyde and the amide compound is selected from among those compounds having the structure IIa:

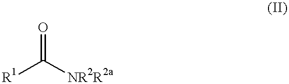

wherein R¹ is hydrocarbyl, substituted hydrocarbyl, or —NR³R⁴, R² and R^{2a} are independently hydrogen, hydrocarbyl, or substituted hydrocarbyl, provided that NR²R^{2a} can be carboxymethylated. Preferably, formalin is selected as the formaldehyde source compound; R¹ is alkyl or NR³R⁴; R² and R³ are independently hydrogen, alkyl, hydroxymethyl, amidomethyl, phosphonomethyl, carboxymethyl, or an ester or salt of carboxymethyl or phosphonomethyl; and R^{2a} and R⁴ are independently hydrogen, hydroxymethyl or another substituent which is hydrolyzable under the carboxymethylation reaction conditions. More preferably, R¹ is methyl, ethyl, isopropyl, or NR³R⁴; R² and R³ are independently hydrogen, methyl, ethyl, isopropyl, hydroxymethyl, carboxymethyl, phosphonomethyl or an ester or salt of carboxymethyl or phosphonomethyl; and R^{2a} and R⁴ are independently hydrogen or hydroxymethyl. Most preferably, R¹ is methyl or NR³R⁴; R² and R³ are independently hydrogen, methyl, hydroxymethyl, carboxymethyl, phosphonomethyl, or an ester or salt of carboxymethyl or phosphonomethyl; and R^{2a} and R⁴ are independently hydrogen or hydroxymethyl. Exemplary amide compounds thus include acetamide; urea; N-alkyl, N-phosphonomethyl, and N-carboxymethyl substituted acetamides; esters and salts of N-phosphonomethyl and N-carboxymethyl substituted acetamides; N,N′-dialkyl, N,N′-diphosphonomethyl, and N,N′-dicarboxymethyl substituted ureas; esters and salts of N,N′-diphosphonomethyl and N,N′-dicarboxymethyl substituted ureas; and amide equivalent compounds selected from the group consisting of:

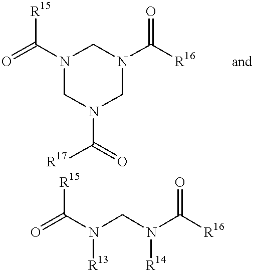

wherein R¹³ and R¹⁴ are independently hydrogen, hydroxymethyl, alkyl, carboxymethyl, phosphonomethyl, or an ester or salt of carboxymethyl or phosphonomethyl; and R¹⁵, R¹⁶ and R¹⁷ are independently alkyl or NR³R⁴. Preferred alkyl substituents for any of R¹³, R¹⁴ R¹⁵, R¹⁶ and R¹⁷ are methyl, ethyl, and isopropyl.

The sequence used to convert the N-acyl amino carboxylic acid product to N-(phosphonomethyl)glycine is dependent upon the amide compound used in the process to produce the N-acyl amino carboxylic acid. Frequently, however, the N-acyl group is hydrolyzed or otherwise removed from the N-acyl amino carboxylic acid product and, if the amide compound did not contain an N-phosphonomethyl substituent, the reaction product is phosphonomethylated either simultaneously with or subsequent to deacylation to remove the N-acyl substituent. Additional steps which can be employed include oxidative cleavage, metallic promoter recycle, and carboxymethylation catalyst recycle, as described elsewhere herein or as known in the art.

### Preparation of N-(phosphonomethyl)glycine from Acetamide

A preferred method for the preparation of N-(phosphonomethyl)glycine using acetamide as the amide compound is depicted in Reaction Scheme 6.

As depicted, one equivalent of acetamide (VII) is reacted with two equivalents each of carbon monoxide and formaldehyde in the presence of a supported noble metal promoter, a carboxymethylation catalyst precursor, and solvent. Under these conditions the acetamide is believed to be protonated with the carboxymethylation catalyst precursor to form BH+[Co(CO)₄]⁻. The reaction produces N-acetyl iminodiacetic acid (XVI) and a carboxymethylation catalyst reaction product (BH+[Co(CO)₄]⁻ wherein “B” is acetamide).

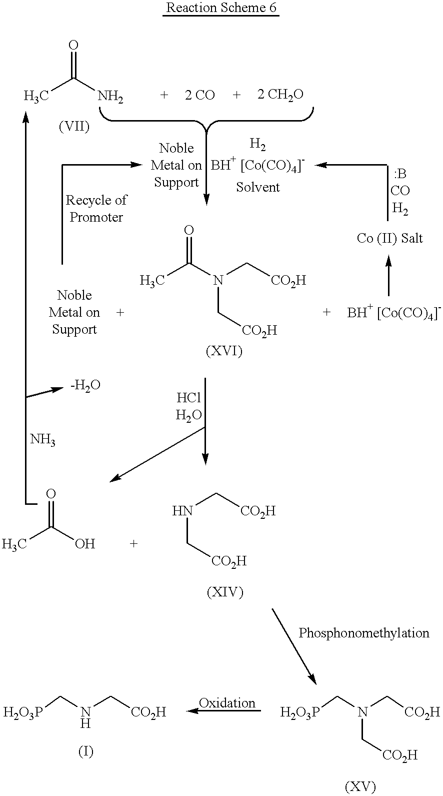

In the presence of water and an acid such as hydrochloric acid, N-acetyl iminodiacetic acid (XVI) is hydrolyzed to form iminodiacetic acid (XIV) and acetic acid. The separated iminodiacetic acid (XIV) is reacted in a phosphonomethylation reaction with formaldehyde and H₃PO₃, PCl₃ or other H₃PO₃ source to produce N-(phosphonomethyl) iminodiacetic acid (XV) which is oxidized, for example, in the presence of a carbon or platinum on carbon catalyst to yield N-(phosphonomethyl)glycine (I). This oxidation is described by, for example, Ebner et al. in PCT/US99/03402.

The supported noble metal promoter used to promote (i.e., accelerate) the carboxymethylation step of the reaction can be optionally recycled by separation from the reaction mixture and returned to a new carboxymethylation reaction as previously described above in Section C.

In a preferred embodiment, the supported noble metal promoter in the carboxymethylation reaction mixture is palladium on carbon. Palladium on carbon and similar supported noble metals as disclosed above, when used as a promoter (i.e., accelerator) of the carboxymethylation reaction, have been found to provide the following surprising and significant results:

1) certain ratios of supported noble metal promoter to the carboxymethylation reaction mixture as described herein significantly increase the rate of reaction compared to unpromoted carboxymethylation reactions (by, it is theorized and believed, activating hydrogen for the carboxymethylation);

2) certain ratios of supported noble metal promoter added to the carboxymethylation reaction mixture as described herein achieve a rate of carboxymethylation reaction and a yield of N-acetyl iminodiacetic acid (XVI) comparable to that achieved using a strong acid co-catalyst such as sulfuric acid or hydrochloric acid;

3) use of certain ratios of supported noble metal promoter added to the carboxymethylation reaction mixture as described herein afford markedly reduced levels of the by-products, iminodiacetic acid (IDA), N-methyl iminodiacetic acid (MeIDA), glycine, and ammonia, and higher purity N-acyl iminodiacetic acid by eliminating the need to use strong acids that cause hydrolysis and side reactions of starting materials, intermediates, and the N-acyl iminodiacetic acid product when acetamide is used; and

4) use of a supported noble metal promoter does not cause the carboxymethylation reaction to proceed in the absence of the cobalt carboxymethylation catalyst.

The cobalt used in the carboxymethylation step of the reaction may be recovered as a cobalt(II) salt as previously described above in Section C. In addition, regenerating the cobalt(II) salt in the presence of acetamide (:B), carbon monoxide and hydrogen may result in the formation of BH+[Co(CO)₄]⁻ which may be recycled to the carboxymethylation reaction mixture.

Similarly, the acetic acid, which is generated by the hydrolysis of N-acetyl iminodiacetic acid (XVI) to iminodiacetic acid (XIV), may optionally be reacted with ammonia to form acetamide and recycled for use as a starting material in the carboxymethylation reaction. As a result, increased efficiency is achieved in the use of raw materials by converting ammonia, carbon monoxide and formaldehyde into iminodiacetic acid.

In a preferred embodiment in which the amide compound is acetamide or an acetamide equivalent (that is, a composition which can be hydrolyzed to acetamide under the carboxymethylation reaction conditions), the reaction mixture for the carboxymethylation reaction contains acetic acid as an organic acid co-catalyst. Alternatively or simultaneously, a strong mineral acid such as sulfuric acid or a strong organic acid such as methanesulfonic acid may be added. As a result, high yields of N-acetyl iminodiacetic acid (XVI) can be obtained at relatively high payloads.

In accordance with the present invention, the molar ratio of acetic acid to cobalt generally ranges from about 2:1 to about 60:1, preferably from about 7:1 to about 55:1, and still more preferably from about 10:1 to about 50:1. At relatively lower pressures, for example, pressures less than about 1,800 psi (12,500 kPa), the molar ratio of acetic acid to cobalt generally ranges from about 2:1 to about 20:1, preferably from about 7:1 to about 15:1, and still more preferably from about 11:1 to about 13:1. At intermediate pressures, for example, pressures within the range of about 1,800 to about 2,500 psi (12,500 to about 17,250 kPa), the molar ratio of acetic acid to cobalt generally ranges from about 2:1 to about 45:1, preferably from about 8:1 to about 30:1, and still more preferably from about 10:1 to about 20:1. At relatively high pressures, for example, pressures of at least about 2,500 psi (17,250 kPa), the molar ratio of acetic acid to cobalt generally ranges from about 4:1 to about 60:1, preferably from about 8:1 to about 55:1, and still more preferably from about 10:1 to about 50:1. The effect of using acetic acid on the carboxymethylation of acetamide (VII), is disclosed in PCT application publication number WO 9835930 which shows the percent yield of N-acetyl iminodiacetic acid (XVI) based on starting amount of acetamide (VII) under different reaction conditions of pressure, payload, solvent, added water, amount of Co₂(CO)₈ catalyst precursor, and added acetic acid co-catalyst.

The experimental data in PCT application publication number WO 9835930 further suggests that yield is improved when acetic acid is used as a co-catalyst in the carboxymethylation of acetamide (VII) when water is present in the reaction mixture. Typically, the molar ratio of water to acetamide starting material is from about 1:1 to about 5:1, preferably from about 2:1 to about 4:1, and more preferably from about 3.2:1 to about 3.8:1.

The yield of N-acetyl iminodiacetic acid increases with increasing pressure as disclosed in PCT application publication number WO 9835930. Accordingly, if lesser catalyst loads or greater payloads are desired for the reaction mixture, it is preferred that the carboxymethylation reaction of acetamide be carried out at a pressure of at least about 500 psi (3,500 kPa), more preferably at least about 1,500 psi (10,500 kPa), and most preferably from about 3,000 psi to about 3,500 psi (21,000 kPa to 24,000 kPa).

PCT application publication number WO 9835930 further illustrates that an increase in pressure allows for an increase in payload. For a given catalyst load, increasing pressure permits an increase in payload while maintaining high, commercially acceptable yields of N-acetyl iminodiacetic acid. Thus, for example, increasing pressure from 1,500 psi (10,340 kPa) to 3,200 psi (22,000 kPa) allows the payload to be doubled without a loss in yield, whereas a doubling of payload at 1,500 psi (10,340 kPa) results in a significant loss of yield.

An alternative route for the preparation of N-(phosphonomethyl)glycine (I) from acetamide VII is illustrated in Reaction Scheme 7:

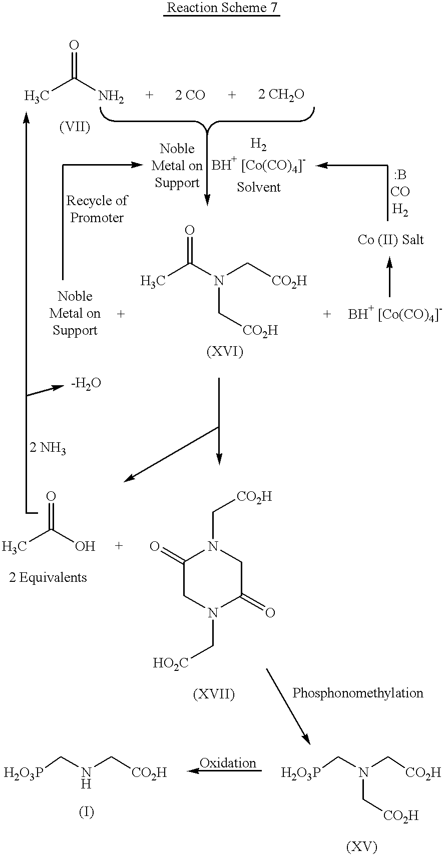

In general, the sequence of reactions in Reaction Scheme 7 is the same as those in Reaction Scheme 6 except that N-acetyl iminodiacetic acid (XVI) is deacylated-cyclized to form 1,4-di(carboxymethyl)-2,5-diketopiperazine (XVII) which is then directly phosphonomethylated in the same manner that iminodiacetic acid (XIV) is phosphonomethylated in Reaction Scheme 6.

A third alternative reaction scheme for the preparation of N-(phosphonomethyl)glycine (I) from acetamide VII is depicted in Reaction Scheme 8:

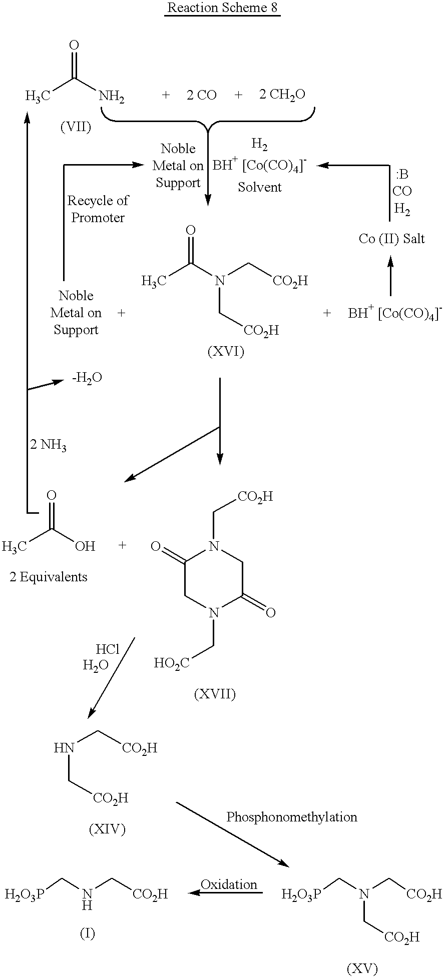

In general, the sequence of reactions in Reaction Scheme 8 is the same as those in Reaction Scheme 7 except that 1,4-di(carboxymethyl)-2,5-diketopiperazine (XVII) is hydrolyzed using water and an acid such as hydrochloric acid to iminodiacetic acid (XIV) which is then phosphonomethylated as described in Reaction Scheme 6.

A fourth alternative reaction scheme for the preparation of N-(phosphonomethyl)glycine (1) from acetamide VII is depicted in Reaction Scheme 9. As depicted in Reaction Scheme 9 below, one equivalent of acetamide VII is reacted with one equivalent each of carbon monoxide and formaldehyde in the presence of a supported noble metal promoter, carboxymethylation catalyst precursor and solvent to yield N-acetyl glycine XVIII. In this reaction sequence, recycle of the supported noble metal promoter and recycle and regeneration of the cobalt(II) salt are as described in connection with Reaction Scheme 6.

In contrast to Reaction Scheme 6, however, N-acetyl glycine (XVII) is reacted with formaldehyde and H₃PO₃, PCl₃ or other H₃PO₃ source to produce N-(phosphonomethyl)-N-acetyl glycine (XIX) which is hydrolyzed using water and an acid such as hydrochloric acid to produce N-(phosphonomethyl)glycine (I) and acetic acid. Acetic acid which is produced in the hydrolysis step can be reacted with ammonia to regenerate acetamide for the carboxymethylation step.

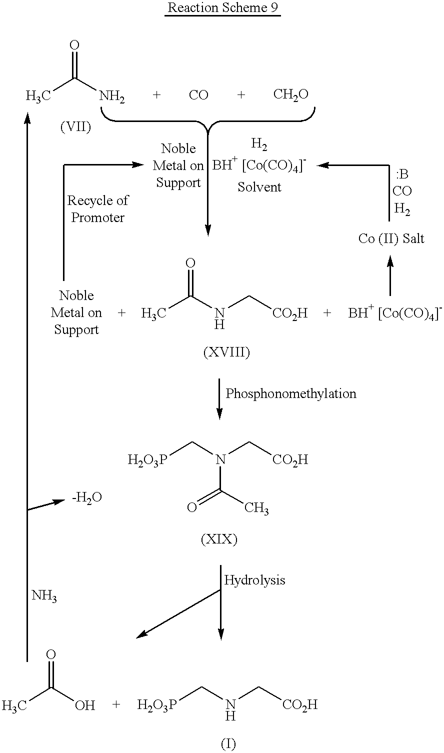

A fifth alternative reaction scheme for the preparation of N-(phosphonomethyl)glycine (I) from acetamide (VII) is depicted in Reaction Scheme 9a:

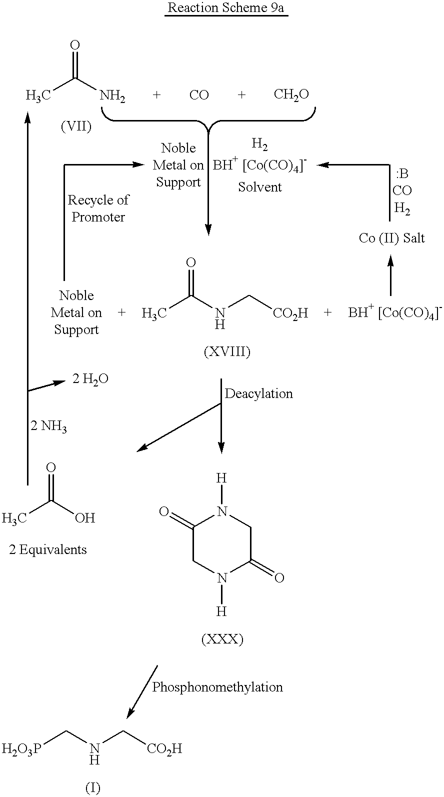

The sequence of reactions in Reaction Scheme 9a is comparable to those set forth in Reaction Scheme 9 except that N-acetyl glycine (XVIII) is deacylated to form 2,5-diketopiperazine (XXX). 2,5-diketopiperazine XXX is then reacted with formaldehyde and H₃PO₃, PCl₃ or other H₃PO₃ source to produce N-(phosphonomethyl)glycine (I) and acetic acid. Acetic acid which is produced in the hydrolysis step can be reacted with ammonia to regenerate acetamide for the carboxymethylation step.

Instead of starting with acetamide in the foregoing reaction schemes, an acetamide equivalent may be used. As used herein, an acetamide equivalent is a composition which, upon hydrolysis, yields acetamide or hydroxymethyl acetamide. Examples of acetamide equivalents include the following compositions:

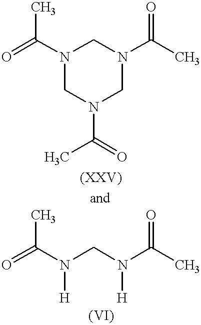

Thus, for example, these compounds may be substituted for acetamide in any one of Reaction Schemes 6, 7, 8, 9 and 9a.

### Preparation of N-(phosphonomethyl)glycine from N-methyl acetamide

A preferred method for the preparation of N-(phosphonomethyl)glycine using N-methyl acetamide as the carbamoyl compound is depicted in Reaction Scheme 10.

As depicted, one equivalent of N-methyl acetamide (IX) is reacted with one equivalent each of carbon monoxide and formaldehyde in the presence of a supported noble metal promoter, a carboxymethylation catalyst precursor and solvent to yield N-acetyl sarcosine. In the presence of water and an acid such as hydrochloric acid, N-acetyl sarcosine is hydrolyzed to sarcosine (XXIII) and acetic acid. Sarcosine (XXIII) is reacted with formaldehyde and H₃PO₃, PCl₃ or other H₃PO₃ source to produce N-(phosphonomethyl)-N-methyl-glycine (XXI) which is oxidized in the presence of a platinum catalyst and oxygen to N-(phosphonomethyl)glycine (I).

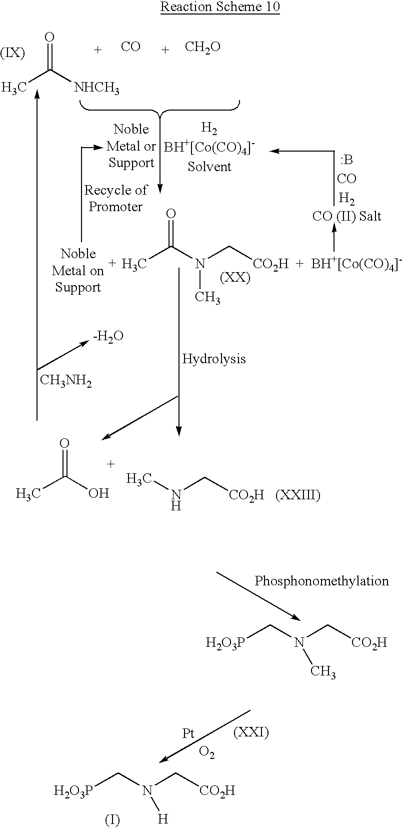

Similar to the preparation of glyphosate from acetamide as described in connection with Reaction Scheme 6, the carboxymethylation catalyst reaction product (BH⁺[Co(CO)₄]⁻ wherein “B” is N-methyl acetamide) is optionally recycled and then regenerated in the presence of N-methyl acetamide.

Also, acetic acid which is generated by the hydrolysis of N-acetyl sarcosine to sarcosine (XXIII) is optionally reacted with methylamine to form N-methyl acetamide and recycled for use as a starting material in the carboxymethylation reaction.

An alternative route for the preparation of N-(phosphonomethyl)glycine (I) from N-methyl acetamide (IX) is illustrated in Reaction Scheme 11. In general, the sequence of reactions in Reaction Scheme 11 is the same as those in Reaction Scheme 10 except that N-acetyl sarcosine is deacylated to form 1,4-dimethyl-2,5-diketopiperazine (XXV). 1,4-dimethyl-2,5-diketopiperazine (XXV) is then directly phosphonomethylated in the same manner that sarcosine (XXIII) is phosphonomethylated in Reaction Scheme 10.

Alternatively, 1,4-dimethyl-2,5-diketopiperazine (XXV) may be hydrolyzed to sarcosine (XXIII) and phosphonomethylated as described in connection with Reaction Scheme 10.

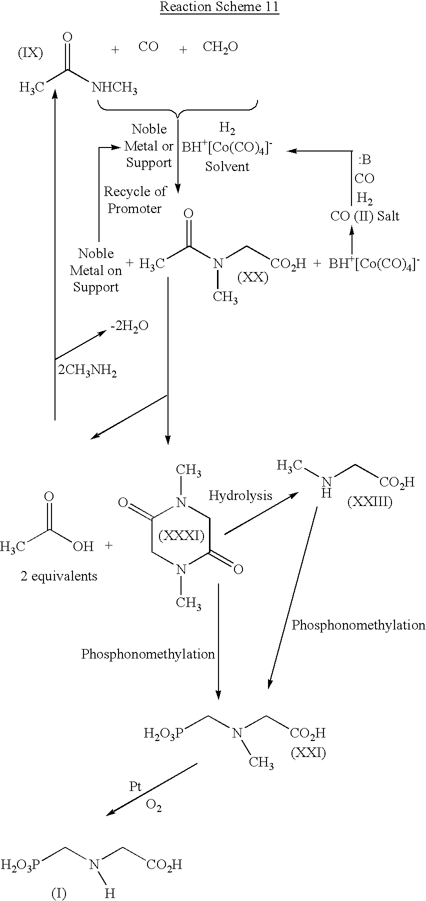

A third alternative reaction scheme for the preparation of N-(phosphonomethyl)glycine (I) from N-methyl acetamide (IX) is depicted in Reaction Scheme 12:

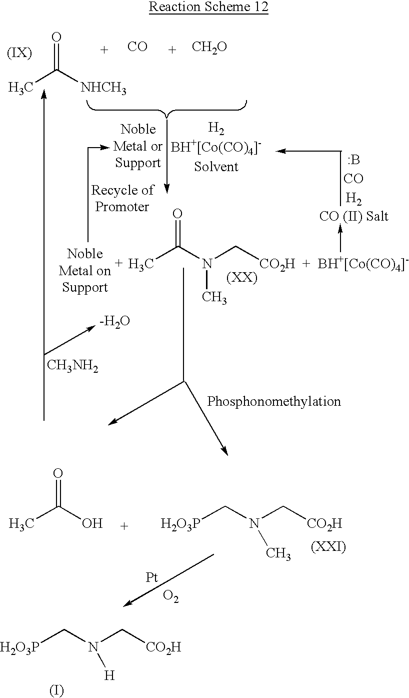

As depicted, the carboxymethylation step of Reaction Scheme 12 is the same as the carboxymethylation step of Reaction Schemes 10 and 11. In Reaction Scheme 12, however, N-acetyl sarcosine is reacted with formaldehyde and H₃PO₃, PCl₃ or other H₃PO₃ source to produce N-phosphonomethyl-N-methyl-glycine (XXI) which is oxidized in the presence of a platinum catalyst and oxygen to N-(phosphonomethyl)glycine (I) and acetic acid. The acetic acid is then reacted with methylamine to yield the N-methyl acetamide starting material.

### Preparation of N-(phosphonomethyl)glycine from N-acetyl glycine (XVIII)

A preferred method for the preparation of N-(phosphonomethyl)glycine starting from N-acetyl glycine (XVIII) is depicted in Reaction Schemes 13 and 14. N-acetyl glycine (XVIII) is carboxymethylated in the presence of a supported noble metal promoter to yield N-acetyl iminodiacetic acid (XVI) which is then converted to N-(phosphonomethyl)glycine (I), for example, as described in Reaction Schemes 6, 7, and 8.

Acetic acid is produced as a hydrolysis product in each of Reaction Schemes 13 and 14. The acetic acid is optionally reacted with ammonia to regenerate acetamide (VII) which can then be carboxymethylated to make N-acetyl glycine (XVIII).

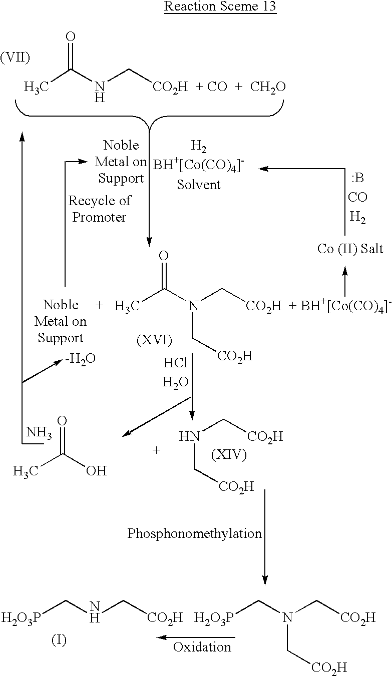

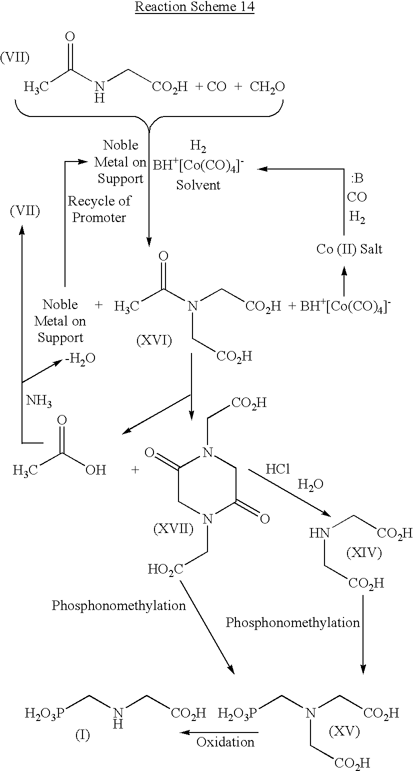

### Preparation of N-(phosphonomethyl)glycine from N-methyl acetamide equivalent

A preferred method for the preparation of N-phosphonomethyl)glycine using VIII, which is an N-methyl acetamide equivalent, is depicted in Reaction Schemes 15 and 16. Thus, VIII is carboxymethylated to form N-methyl-N-acetyl glycine (XX) which is converted to N-(phosphonomethyl)glycine (I), for example, as described in Reaction Schemes 10, 11 and 12.

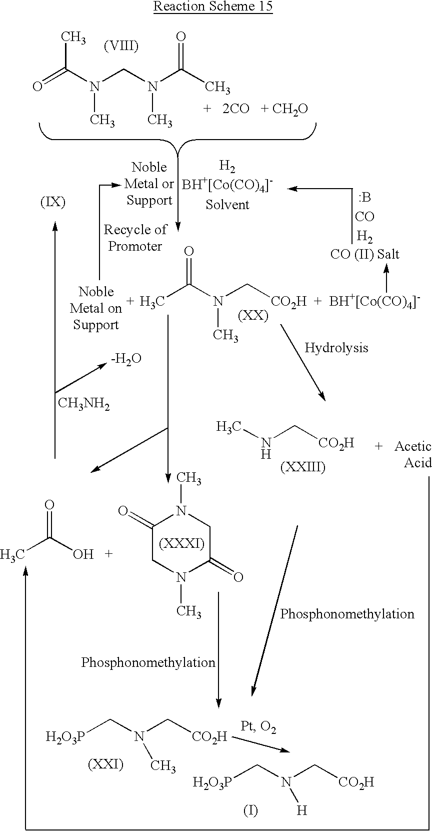

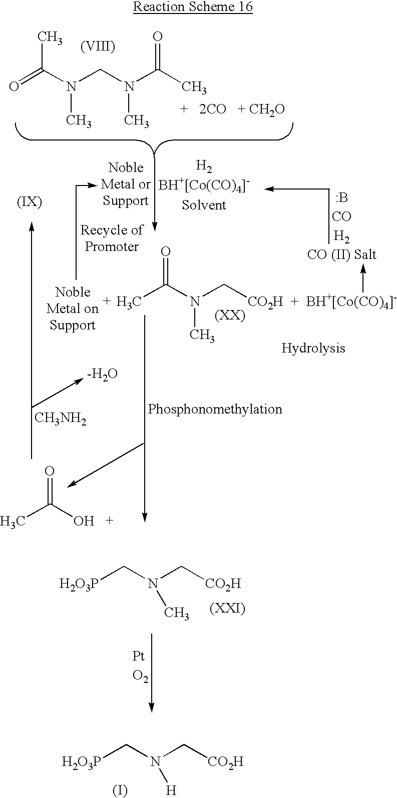

### Preparation of N-(phosphonomethyl)glycine from Urea

A preferred method for the preparation of N-(phosphonomethyl)glycine from urea is depicted in Reaction Scheme 17:

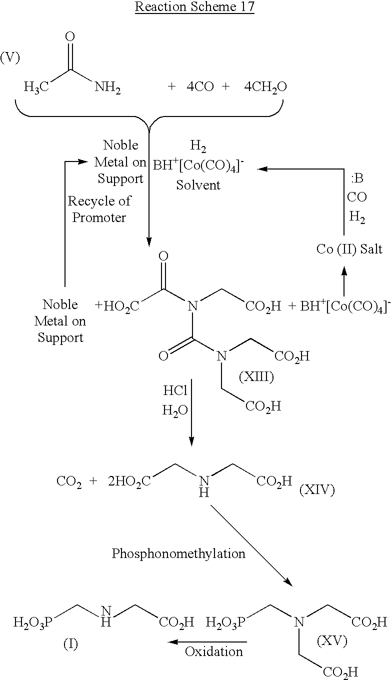

As depicted, one equivalent of urea (V) is reacted with four equivalents each of carbon monoxide and formaldehyde in the presence of a supported noble metal promoter, a carboxymethylation catalyst precursor and solvent. In contrast to Reaction Scheme 6, urea (V) is reacted in Reaction Scheme 17 with the carboxymethylation catalyst precursor in the absence of formalin to form (BH⁺[Co(CO)₄]⁻ wherein B is urea).

The products of the carboxymethylation reaction are the tetraacid (XIII) and the carboxymethylation catalyst reaction product (BH⁺[Co(CO)₄]⁻ wherein “B” is urea). Tetraacid (XIII) is hydrolyzed to 2 equivalents of iminodiacetic acid (XIV) and carbon dioxide. Iminodiacetic acid (XIV) is converted to N-(phosphonomethyl)glycine (I), for example, as described in connection with Reaction Schemes 6 and 8.

### Preparation of N-(phosphonomethyl)glycine from N,N-Dimethylurea

A preferred method for the preparation of N-(phosphonomethyl)glycine from N,N-dimethylurea is depicted in Reaction Scheme 18.

As depicted below, one equivalent of N,N-dimethylurea (X) is reacted with two equivalents each of carbon monoxide and formaldehyde in the presence of a carboxymethylation catalyst precursor, a supported noble metal promoter and solvent. Similar to Reaction Scheme 17, N,N-dimethylurea (X) is reacted with the carboxymethylation catalyst precursor in the absence of formalin to form (BH+[Co(CO)₄]⁻) wherein BH⁺ is the protonated N,N′-dimethylurea (X).

The products of the carboxymethylation reaction are the diacid (XXII) and the carboxymethylation catalyst reaction product (BH+[Co(CO)₄]⁻ wherein B is N,N′-dimethylurea). Diacid (XXII) is hydrolyzed to 2 equivalents of sarcosine (XXIII) and carbon dioxide. The sarcosine (XXIII) is converted to N-(phosphonomethyl)glycine (I), for example, as described in connection with Reaction Schemes 10 and 15.

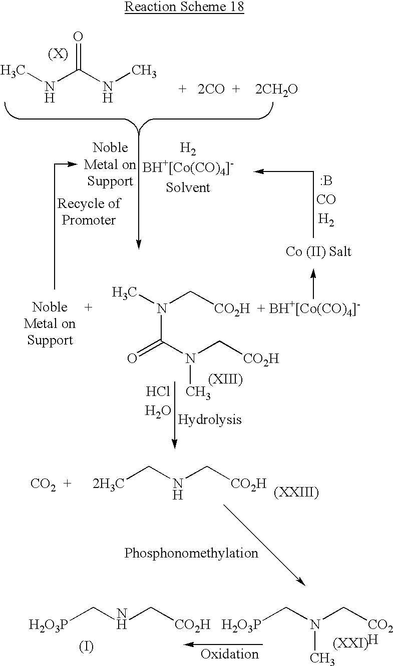

An alternative method for the preparation of N-(phosphonomethyl)glycine (I) from N,N′-dimethylurea (X) is depicted in Reaction Scheme 19:

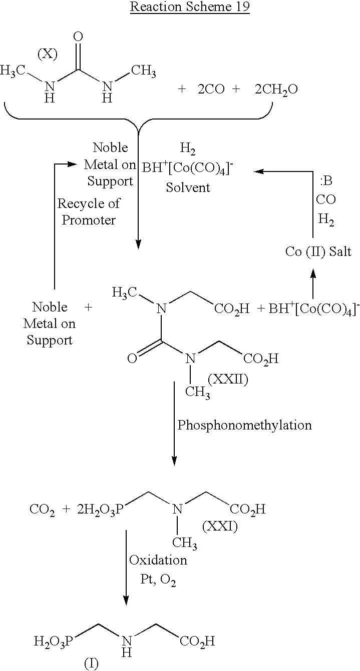

As depicted, the carboxymethylation reaction is carried out as described in Reaction Scheme 18 to yield diacid (XXII), the supported noble metal promoter, and the carboxymethylation catalyst reaction product (BH+[Co(CO)₄]⁻ wherein “B” is N,N′-dimethylurea). In this reaction scheme, however, diacid (XXII) is reacted with formaldehyde and H₃PO₃, PCl₃ or another H₃PO₃ source to produce N-phosphonomethyl-N-methyl glycine (XXI) which is oxidized in the presence of a platinum catalyst and oxygen to N-(phosphonomethyl)glycine (I).

### Preparation of N-(phosphonomethyl)glycine from bis-phosphonomethylurea

A preferred method for the preparation of N-(phosphonomethyl)glycine from bis-phosphonomethylurea (XII) is depicted in Reaction Scheme 20:

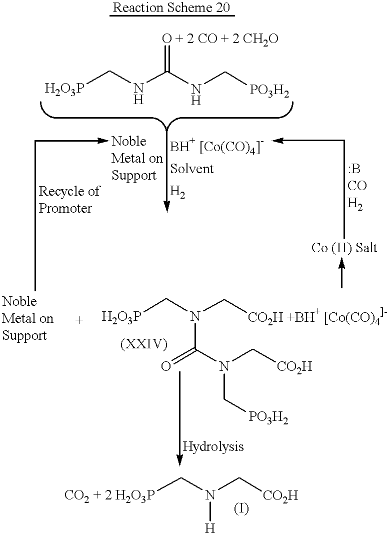

As depicted, one equivalent of bis-phosphonomethylurea (XII) is reacted with two equivalents each of carbon monoxide and formaldehyde in the presence of a carboxymethylation catalyst precursor, a supported noble metal promoter and solvent. In this reaction scheme bis-phosphonomethylurea (XII) is reacted with the carboxymethylation catalyst precursor in the absence of formalin to form BH⁺[Co(CO)₄]⁻ wherein BH⁺ is the protonated bis-phosphonomethylurea (XII).

The products of the carboxymethylation reaction are XXIV and the carboxymethylation catalyst reaction product (BH⁺[Co(CO)₄]⁻ wherein “B” is bisphosphonomethylurea). XXIV is reacted with formaldehyde and H₃PO₃, PCl₃ or other H₃PO₃ source to produce N-phosphonomethyl)glycine (I).

### Preparation of N-(phosphonomethyl)glycine from N-acetyl-N-(phosphonomethyl)amine

A preferred method for the preparation of N-(phosphonomethyl)glycine (I) from N-acetyl-N-(phosphonomethyl)amine (XI) is depicted in Reaction Scheme 21:

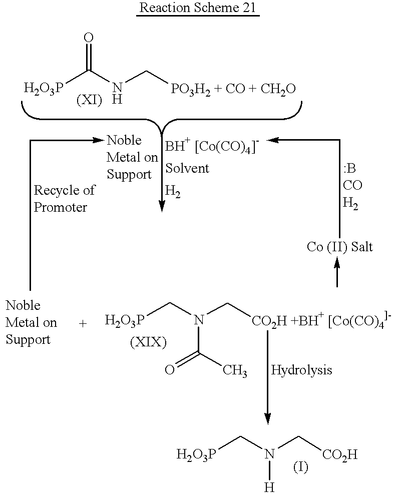

As depicted, one equivalent of N-acetyl-N-(phosphonomethyl)amine (XI) is reacted with one equivalent each of carbon monoxide and formaldehyde in the presence of a supported noble metal promoter, a carboxymethylation catalyst precursor and solvent. In this reaction scheme N-acetyl-N-(phosphonomethyl)amine is reacted with the carboxymethylation catalyst precursor in the absence of formalin to form (BH⁺[Co(CO)₄]⁻) wherein BH⁺ is the protonated N-acetyl-N-(phosphonomethyl)glycine (XI).

The products of the carboxymethylation reaction are XIX and the carboxymethylation catalyst reaction product (BH⁺[Co(CO)₄]⁻) wherein “B” is N-acetyl-N-(phosphonomethyl)amine. XIX is reacted with formaldehyde and H₃PO₃, PCl₃ or other H₃PO₃ source to produce N-(phosphonomethyl)glycine (I).

Definitions

The following definitions are provided in order to aid the reader in understanding the detailed description of the present invention:

“Glyphosate” means N-(phosphonomethyl)glycine in acid form or any of its salt or ester forms.

“Hydrocarbyl” means a group composed of carbon and hydrogen. This definition includes alkyl, alkenyl, and alkynyl groups which are each straight chain, branched chain, or cyclic hydrocarbons from one to about twenty carbons. Also included in this definition are aryl groups composed of carbon and hydrogen. Hydrocarbyl therefore includes, for example, methyl, ethyl, propyl, butyl, pentyl, hexyl, octyl, decyl, dodecyl, tetradecyl, bexadecyl, octadecyl, eicosyl, cyclopropyl, cyclobutyl, cyclopentyl, cyclohexyl, methylcyclopentyl, ethenyl, propenyl, butenyl, pentenyl, hexenyl, ethynyl, propynyl, butynyl, pentynyl, hexynyl, phenyl, naphthyl, anthracenyl, benzyl, and isomers thereof.

“Substituted hydrocarbyl” means a hydrocarbyl group in which one or more hydrogens has been replaced by a heteroatom-containing group. Such substituent groups include, for example, halo, oxo, heterocyclyl, alkoxy, hydroxy, aryloxy, —NO₂, amino, alkylamino, or amido. When the substituent group is oxo, the substituted hydrocarbyl can be, for example, an acyl group.

“Heteroatom” means an atom of any element other than carbon or hydrogen which is capable of forming chemical bonds.

“Heterocycle” means a saturated or unsaturated mono- or multi-ring carbocycle wherein one or more carbon atoms is replaced by N, S, P, or O. This includes, for example, the following structures:

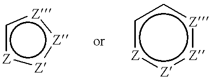

wherein Z, Z′, Z″, or Z′″ is C, S, P, O, or N, with the proviso that one of Z, Z′, Z″, or Z′″ is other than carbon, but is not O or S when attached to another Z atom by a double bond or when attached to another O or S atom. Furthermore, the optional substituents are understood to be attached to Z, Z′, Z″, or Z″′ only when each is C. The point of attachment to the molecule of interest can be at the heteroatom or elsewhere within the ring.

“Halogen” or “halo” means a fluoro, chloro, bromo, or iodo group.

“Amide” means a group containing a fully saturated nitrogen atom attached by a single bond to a carbonyl moiety.

“Carboxymethyl” means a group containing a carboxylate moiety attached by the carboxylate carbon atom to a saturated carbon atom which in turn is attached to the molecule of interest.

“Carboxymethylation catalyst” means a catalyst which is useful in carbonylation reactions, and particularly in carboxymethylation reactions.

“Carboxymethylation” means the introduction of a substituted or unsubstituted carboxymethyl group into the molecule of interest.

“Payload” means the mass of starting material divided by the mass of reaction solvent.

“Metallic Promoter” is defined as a promoter of the present invention wherein the promoter contains a metal which is free, complexed by ligands, deposited on an essentially inert support, or present as part of an anion or cation and the metallic promoter is able to accelerate a carboxymethylation reaction catalyzed by a carboxymethylation catalyst precursor, facilitate conversion of a carboxymethylation catalyst precursor to an active catalyst species and wherein the metallic promoter is itself unable to function significantly and directly as a carboxymethylation catalyst in the absence of a carboxymethylation catalyst precursor.

“Supported Metallic Promoter” means a “Metallic Promoter” wherein the metal is a metal in the free state deposited on an essentially inert support.

“Supported Noble Metal Promoter” means a “Supported Metallic Promoter” wherein the metal is a noble metal deposited on an essentially inert support.

“PM” means phosphonomethylation.

“GC” means gas chromatography.

“HPLC” means high pressure liquid chromatography.

“IC” means ion chromatography.

“NMR” means nuclear magnetic resonance spectroscopy.

“MS” means mass spectrometry.

The following examples will illustrate the invention.

### EXAMPLES

In the representative examples below of the carboxymethylation process of the present invention or comparative process examples, either a 300 or 2000 mL stainless steel autoclave equipped with a magnetic stirrer and heating system was employed. All compound numbers are in Roman numerals and reflect the structures which appear in Reaction Schemes 1 through 21. The progress of the reaction was monitored by following the consumption of gas. At the end of each heating period the reaction mixture was cooled to ambient temperature before analysis. N-acetyl iminodiacetic acid (XVI) was quantified by HPLC analysis utilizing an Interaction Ion 310 ion exclusion column at 30° C. and UV absorption detection at 210 nm. The 0.04 N H₂SO₄ mobile phase pumped at 0.5 mL/min. gave retention times from 4.6-4.8 min. for the N-acetyl iminodiacetic acid (XVI). All yields are based on moles of specific acylamide charged.

### Comparative Example 1

This comparative example was designed as a baseline experiment to demonstrate a typical result obtained in the absence of a supported noble metal promoter.

A 300 mL autoclave was charged with acetamide (VII) (11.8 g, 0.2 mole), 95% paraformaldehyde (13.6 g, 0.43 mole), acetic acid (4.0 g, 0.067 mole), water (12.9 g, 0.72 mole), THF (90 mL), and CO₂(CO)₈ (0.50 g, 0.0015 mole). The sealed vessel was supplied with 3200 psi of a 95:5 mixture of carbon monoxide and hydrogen and heated with vigorous mechanical stirring to about 110° C. After 30 min at 110° C. the autoclave was cooled and pressure was carefully vented. HPLC analysis of the resulting product mixture showed a 29.8% yield of N-acetyl iminodiacetic acid (XVI) and a 46.6% yield of N-acetyl glycine (XVIII).

### Comparative Example 2

This comparative example demonstrates that sulfuric acid can be used as an effective catalyst, but that the presence of sulfuric acid is not as effective as added supported noble metal promoter on the rate of reaction observed and yield of XVI as compared to Examples 1 and 2 below.

A 300 mL autoclave was charged with acetamide (VII) (11.8 g, 0.2 mole), 95% parafonnaldehyde (13.6 g, 0.43 mole), acetic acid (4.0 g, 0.067 mole), water (12.9 g, 0.72 mole), THF (90 mL), Co₂(CO), (0.50 g, 0.0015 mole), H₂SO₄ (2.00 g, 0.002 mole), and 1.00 g of 10% Pd/C. The sealed vessel was supplied with 3200 psi of a 95:5 mixture of carbon monoxide and hydrogen and heated with vigorous mechanical stirring to about 110° C. After 30 min at 110° C. the autoclave was cooled, and pressure was carefully vented. HPLC analysis of the resulting product mixture showed a 81.0% yield of N-acetyl iminodiacetic acid (XVI) and a 16.4% yield of N-acetyl glycine (XVII).

### Comparative Example 3

This comparative example demonstrates that the use of an unsupported noble metal, palladium wire, results in reduced rate and yield as compared to comparative examples 1 and 2 above and examples 1 and 2 below.

A 300 mL autoclave was charged with acetamide (VII) (11.8 g, 0.2 mole), 95% paraformaldehyde (13.6 g, 0.43 mole), acetic acid (4.0 g, 0.067 mole), water (12.9 g, 0.72 mole), THF (90 mL), Co₂(CO)₈ (0.50 g, 0.0015 mole), and 0.18 g of palladium wire. The sealed vessel was supplied with 3200 psi of a 95:5 mixture of carbon monoxide and hydrogen and heated with vigorous mechanical stirring to about 110° C. After 30 min at 110° C. the autoclave was cooled, and pressure was carefully vented. HPLC analysis of the resulting product mixture showed a 6.1% yield of N-acetyl iminodiacetic acid (XVI) and a 44.0% yield of N-acetyl glycine (XVIII).

### Comparative Example 4

This comparative example demonstrates that the supported noble metal promoter most likely functions as a hydrogen activation promoter. The reaction is slow and yield is relatively poor in the absence of hydrogen as compared to Examples 1-3 below.

A 300 mL autoclave was charged with acetamide (VII) (11.8 g, 0.2 mole), 95% paraformaldehyde (13.6 g, 0.43 mole), acetic acid (4.0 g, 0.067 mole), water (12.9 g, 0.72 mole), THF (90 mL), Co₂(CO)₈ (0.50 g, 0.0015 mole), and 1.01 g of 10% Pd/C. The sealed vessel was supplied with 3200 psi of 100% carbon monoxide and heated with vigorous mechanical stirring to about 110° C. After 30 min at 110° C. the autoclave was cooled and pressure was carefully vented. HPLC analysis of the resulting product mixture showed a 0.8% yield of N-acetyl iminodiacetic acid (XVI) and a 18.2% yield of N-acetyl glycine (XVIII).

### Comparative Example 5

This comparative example demonstrates that the supported noble metal promoter is totally ineffective as a catalyst for carrying out the carboxymethylation reaction as compared to Comparative Examples 1 and 2 above and Examples 1 and 2 below.

A 300 mL autoclave was charged with acetamide (VII) (11.8 g, 0.2 mole), 95% paraformaldehyde (13.6 g, 0.43 mole), acetic acid (4.0 g, 0.067 mole), water (12.9 g, 0.72 mole), THF (90 mL), and 0.50 g of 10% Pd/C. The sealed vessel was supplied with 3200 psi of 100% carbon monoxide and heated with vigorous mechanical stirring to about 110° C. After 30 min at 110° C. the autoclave was cooled and pressure was carefully vented. HPLC analysis of the resulting product mixture showed a 0.0% yield of N-acetyl iminodiacetic acid (XVI) and a 0.0% yield of N-acetyl glycine (XVIII).

### Example 1

This example demonstrates the markedly enhanced rate of reaction observed when a small quantity of a supported noble metal promoter was added to the carboxymethylation reaction mixture of the present invention.

A 300 mL autoclave was charged with acetamide (VII) (11.8 g, 0.2 mole), 95% paraformaldehyde (13.6 g, 0.43 mole), acetic acid (4.0 g, 0.067 mole), water (12.9 g, 0.72 mole), THF (90 mL), CO₂(CO), (0.50 g, 0.0015 mole), and 0.25 g of 10% Pd/C, a supported noble metal promoter. The sealed vessel was supplied with 3200 psi of a 95:5 mixture of carbon monoxide and hydrogen and heated with vigorous mechanical stirring to about 110° C. After 30 min at 110C the autoclave was cooled, and pressure was carefully vented. HPLC analysis of the resulting product mixture showed a 74.7% yield of N-acetyl iminodiacetic acid (XVI) and a 16.0% yield of N-acetyl glycine (XVIII).

### Example 2

This example demonstrates and increased rate of reaction and yield of N-acetyl iminodiacetic acid (XVI) when the quantity of supported noble metal promoter in the carboxymethylation reaction mixture of the present invention is increased.

A 300 mL autoclave was charged with acetamide (VII) (11.8 g, 0.2 mole), 95% paraformaldehyde (13.6 g, 0.43 mole), acetic acid (4.0 g, 0.067 mole), water (12.9 g, 0.72 mole), THF (90 mL), Co₂(CO), (0.50 g, 0.0015 mole), and 1.00 g of 10% Pd/C. The sealed vessel was supplied with 3200 psi of a 95:5 mixture of carbon monoxide and hydrogen and heated with vigorous mechanical stirring to about 110° C. After 30 min at 110° C. the autoclave was cooled, and pressure was carefully vented. HPLC analysis of the resulting product mixture showed a 85.8% yield of N-acetyl iminodiacetic acid (XVI) and a 11.5% yield of N-acetyl glycine (XVI).

### Example 3

This example demonstrates that lower loadings of Pd/C can function effectively as a supported noble metal promoter in the carboxymethylation reaction mixture of the present invention.

A 300 mL autoclave was charged with acetamide (VII) (11.8 g, 0.2 mole), 95% paraformaldehyde (13.6 g, 0.43 mole), acetic acid (4.0 g, 0.067 mole), water (12.9 g, 0.72 mole), THF (90 mL), Co₂(CO)₈ (0.50 g, 0.0015 mole), and 2.03 g of 1% Pd/C. The sealed vessel was supplied with 3200 psi of a 95:5 mixture of carbon monoxide and hydrogen and heated with vigorous mechanical stirring to about 110° C. After 30 min at 110° C. the autoclave was cooled, and pressure was carefully vented. HPLC analysis of the resulting product mixture showed a 72.6% yield of N-acetyl iminodiacetic acid (XVI) and a 21.1% yield of N-acetyl glycine (XVIII).

### Example 4

Example 4, compared to Comparative Example 1 above, shows that the supported noble metal, platinum on carbon, functions effectively as a supported noble metal promoter on the rate of reaction observed and yield of XVI.

A 300 mL autoclave was charged with acetamide (VII) (11.8 g, 0.2 mole), 95% paraformaldehyde (13.6 g, 0.43 mole), acetic acid (4.0 g, 0.067 mole), water (12.9 g, 0.72 mole), THF (90 mL), Co₂(CO)₈ (0.50 g, 0.0015 mole), and 0.50 g of 5% Pt/C. The sealed vessel was supplied with 3200 psi of a 95:5 mixture of carbon monoxide and hydrogen and heated with vigorous mechanical stirring to about 110° C. After 30 min at 110° C. the autoclave was cooled, and pressure was carefully vented. HPLC analysis of the resulting product mixture showed a 43.6% yield of N-acetyl iminodiacetic acid (XVI) and a 41.0% yield of N-acetyl glycine (XVIII).

### Example 5

Example 5, compared to Comparative Example 1 above, shows that the supported noble metal promoter, 10% Pd/C, can be recycled, accelerate a new batch of reactants without loss of activity, and give comparable yields of XVI and XVIII.

Batch 1: A 300 mL autoclave was charged with acetamide (VII) (11.8 g, 0.2 mole), 95% paraformaldehyde (13.6 g, 0.43 mole), acetic acid (4.0 g, 0.067 mole), water (12.9 g, 0.72 mole), THF (90 mL), Co₂(CO)₈ (0.50 g, 0.0015 mole), and 0.26 g of 10% Pd/C, a supported noble metal promoter. The sealed vessel was supplied with 3200 psi of a 95:5 mixture of carbon monoxide and hydrogen and heated with vigorous mechanical stirring to about 110° C. After 30 min at 110° C. the autoclave was cooled, and pressure was carefully vented. HPLC analysis of the resulting product mixture showed a 73.2% yield of N-acetyl iminodiacetic acid (XVI) and a 19.1% yield of N-acetyl glycine (XVII). After allowing the 10% Pd/C to settle, the supernatant was carefully decanted.

Batch 2: The 300 mL autoclave containing 0.25 g of 10% Pd/C from Batch 1, was charged with acetamide (VII) (11.8 g, 0.2 mole), 95% paraformaldehyde (13.6 g, 0.43 mole), acetic acid (4.0 g, 0.067 mole), water (12.9 g, 0.72 mole), THF (90 mL), and Co₂(CO), (0.50 g, 0.0015 mole). The sealed vessel was supplied with 3200 psi of a 95:5 mixture of carbon monoxide and hydrogen and heated with vigorous mechanical stirring to about 110° C. After 30 min at 110° C. the autoclave was cooled, and pressure was carefully vented to give a 80.8% yield of XVI and a 12.3% yield of N-acetyl glycine (XVIII).

### Example 6

Example 6, compared to Comparative Example 1 above, shows that the supported noble metal promoter, 1% Pd/C, loses it activity to accelerate a new batch of reactants in a batch process when recycled.

Batch 1: A 300 mL autoclave was charged with acetamide (VII) (11.8 g, 0.2 mole), 95% paraformaldehyde (13.6 g, 0.43 mole), acetic acid (4.0 g, 0.067 mole), water (12.9 g, 0.72 mole), THF (90 mL), Co2(CO), (0.50 g, 0.0015 mole), and 2.00 g of 1% Pd/C, a supported noble metal promoter. The sealed vessel was supplied with 3200 psi of a 95:5 mixture of carbon monoxide and hydrogen and heated with vigorous mechanical stirring to about 110° C. After 30 min at 110° C. the autoclave was cooled, and pressure was carefully vented to give a 69.9% yield of XVIII and a 22.5% yield of N-acetyl glycine (XVI) by HPLC analysis. After allowing the 1% Pd/C to settle, the supernatant was carefully decanted.

Batch 2: The 300 mL autoclave containing 2.0 g of 1% Pd/C from Batch 1, was charged with acetamide (VII) (11.8 g, 0.2 mole), 95% paraformaldehyde (13.6 g, 0.43 mole), acetic acid (4.0 g, 0.067 mole), water (12.9 g, 0.72 mole), THF (90 mL), and Co₂(CO)₈ (0.50 g, 0.0015 mole). The sealed vessel was supplied with 3200 psi of a 95:5 mixture of carbon monoxide and hydrogen and heated with vigorous mechanical stirring to about 110° C. After 30 min at 110° C. the autoclave was cooled, and pressure was carefully vented. HPLC analysis of the resulting product mixture showed a 28.9% yield of N-acetyl iminodiacetic acid (XVI) and a 43.9% yield of N-acetyl glycine (XVIII).

### Example 7

Example 7 shows that the supported noble metal promoter, 1% Pd/C, can be used in a continuous process to accelerate the reaction of new reactants without loss of activity and to give high yields of XVI with a low level of impurities.

A continuous carboxymethylation system consisting of three 1-L autoclaves, connected in series as reactors/stages 1, 2 and 3, were de-headed to allow addition of the Pd/C co-catalyst. Four stainless steel baskets, each containing 2 g of 1% Pd/C (Aldrich, 4-8 mesh), were wired to and positioned on the cooling coils of each autoclave so that they were in the reaction solution during operation. The reaction was carried out by addition of 35 mL/min total liquid flow into the system. The liquid is a tetrahydrofuran based solution prepared to deliver 55 mmol Acetamide/minute, 132 mmol of formaldehyde source compound from 52 wt % formalin, 1.65 mmol of dicobalt dicarbonyl and 18 mmol of acetic acid. No sulfuric or other mineral acid was added. The run was allowed to proceed for nearly six hours with HPLC analysis of periodical sampling of each reactor (i.e., stage) of the continuous system. Analytical results are summarized in Table 2.

### Table 2.

### Results Summary from a Continuous Carbonylation Reactor with 8 g of 1% Pd/C Per Stage

TABLE 2
Results summary from a Continuous
Carbonylation Reactor with 8 g of 1% Pd/C per stage.

| Stage | 1 | 1 | 1 | 2 | 2 | 2 | 3 | 3 | 3 |
|---|---|---|---|---|---|---|---|---|---|
| Run Time (hr) | 2.9 | 4.9 | 6.0 | 2.9 | 4.9 | 6.0 | 2.9 | 4.9 | 6.0 |
| Product | Percent of Each Product | | | | | | | | |
| VII | 2.0 | 2.1 | 2.1 | 0.0 | 0.0 | 0.0 | 0.0 | 0.0 | 0.0 |
| HMA | 0.9 | 0.7 | 0.6 | 0.0 | 0.0 | 0.0 | 0.0 | 0.0 | 0.0 |
| XVII | 12.4 | 12.4 | 12.2 | 4.3 | 4.5 | 4.5 | 1.8 | 1.9 | 1.9 |
| HMNAG | 2.3 | 2.2 | 2.1 | 0.6 | 0.7 | 0.7 | 0.5 | 0.5 | 0.6 |
| XVI | 82.0 | 82.1 | 82.5 | 94.7 | 94.3 | 94.2 | 97.0 | 96.9 | 96.8 |
| XIV | 0.1 | 0.1 | 0.1 | 0.0 | 0.1 | 0.0 | 0.1 | 0.1 | 0.1 |
| MeIDA | 0.1 | 0.3 | 0.3 | 0.3 | 0.5 | 0.4 | 0.6 | 0.6 | 0.6 |
| Gly | 0.1 | 0.1 | 0.1 | 0.0 | 0. | 0.0 | 0.0 | 0.0 | 0.0 |
| NH₃ | 0.1 | 0.1 | 0.2 | 0.0 | 0. | 0.0 | 0.0 | 0.0 | 0.0 |

HMA is N-hyroxymethylacetamide; HMNAG is N-hydroxymethyl-glycine; MeIDA is N-hyroxymethyl iminodiacetic acid; Gly is glycine.

### Example 8

Example 8 illustrates the advantage of using a supported noble metal promoter, 1% Pd/C, in a continuous process without added sulfuric acid to accelerate the reaction of new reactants. As demonstrated in the results below, the use of sulfuric acid caused hydrolysis and side reactions as compared to Run 3 in which a strong acid was not used. In all cases, the metallic promoter proceeded to accelerate the reaction as compared to Comparative Example 1 above, and to produce high yields of N-acyl iminodiacetic acid XVI with lower level of impurities. Three continuous carboxymethylation reaction runs were conducted as follows:

Run 1 and Run 2: A continuous carboxymethylation reactor system consisting of three 1-L autoclaves connected in series were de-headed to allow addition of the Pd/C co-catalyst. Four stainless steel baskets, each containing 2 g of 1% Pd/C (Aldrich, 4-8 mesh), were wired to and positioned on the cooling coils of each autoclave so that they were in the reaction solution during operation. The reaction was carried out by introducing 35 mL/min total liquid flow into the system. The liquid comprised a tetrahydrofuran based solution prepared to deliver acetamide (55 mmol/minute), formaldehyde (132 mmol from 52 wt % formalin as a formaldehyde source compound), dicobaltoctacarbonyl (1.65 mmol), sulfuric acid (1.65 mmol), and acetic acid (18 mmol). Each run was allowed to proceed for nearly six hours with analysis of each reactor (i.e., stage) of the continuous system. Analytical results are summarized in Table 3.

Run 3: The conditions and overall procedure was identical to that of Runs 1 and 2 without introducing sulfuric acid to the carboxymethylation reaction system. Analytical results are summarized in Table 3.

TABLE 3
Results for Example 8
All runs with 8 g of 1% Pd/C per stage and for six hours.

| Stage | 1 | 2 | 3 | 1 | 2 | 3 | 1 | 2 | 3 |
|---|---|---|---|---|---|---|---|---|---|
| Run # | 1 | | | 2 | | | 3 | | |
| Product | Percent of Each Product | | | | | | | | |
| VII | 0.7 | 0.0 | 0.0 | 0.5 | 0.0 | 0.0 | 2.1 | 0.0 | 0.0 |
| HMA | 0.3 | 0.0 | 0.0 | 0.4 | 0.0 | 0.0 | 0.7 | 0.0 | 0.0 |
| XVII | 9.2 | 2.4 | 1.1 | 7.8 | 1.6 | 0.7 | 12.3 | 4.4 | 1.9 |
| HMNAG | 1.4 | 0.2 | 0.1 | 1.4 | 0.2 | 0.1 | 2.2 | 0.7 | 0.5 |
| XVI | 86.4 | 94.9 | 96.2 | 88.4 | 96.4 | 97.4 | 82.2 | 94.4 | 96.9 |
| XIV | 0.4 | 1.0 | 1.2 | 0.3 | 0.3 | 0.3 | 0.1 | 0.1 | 0.1 |
| MeIDA | 0.8 | 1.2 | 1.3 | 0.8 | 1.4 | 1.4 | 0.2 | 0.4 | 0.6 |
| Gly | 0.2 | 0.1 | 0.1 | 0.1 | 0.1 | 0.0 | 0.1 | 0.0 | 0.0 |
| NH₃ | 0.6 | 0.1 | 0.0 | 0.3 | 0.0 | 0.0 | 0.1 | 0.0 | 0.0 |

HMA is N-hyroxymethylacetamide; HMNAG is N-hydroxymethyl-glycine; MeIDA is N-hyroxymethyl iminodiacetic acid; Gly is glycine.

### Example 9

This example demonstrates the recovery of a cobalt(II) salt from a carboxymethylation product mixture.

After filtering to remove the Pd/C noble metal promoter, a distillation apparatus was charged with a XVI product stream such as that described in Example 2. The distillate bottoms was heated to a temperature of 90° C. and a distillate with a vapor temperature of 85° C. was collected in a receiving flask. Anhydrous DME was then added to the distillation pot at a rate similar to the removal of the 85° C. distillate. After removal of 115 g of 85° C. distillate and addition of 120 g DME, a pink precipitate Co(N-acetyl iminodiacetic acid)₂ was present in the distillation pot. This solid was isolated by filtration. Analysis of this filtrate revealed that it contained ppm levels of cobalt, implying that virtually all of the cobalt can be removed from the carboxymethylation product mixture.

### Example 10

This example demonstrates the regeneration of a carboxymethylation catalyst precursor from a cobalt(II) salt and its reuse in a carboxymethylation reaction.

An autoclave was charged with cobalt acetate tetrahydrate (26.85 g, 0.108 mole) and acetic acid (106 g, 1.77 mole) and pressurized to 2200 psi (15,170 kPa) CO:H₂ (90:10) at 25° C. This mixture was heated to 130° C. for 5 h. Gas uptake was used to indicate that some of the cobalt(II) salt was converted to catalyst precursor.

The regenerated catalyst precursor was transferred, under CO:H₂ pressure, into the autoclave containing CO:H₂ (95:5) at 800 psi (5517 kPa), acetamide (VII)(29.5 g, 0.5 mole), 95% paraformaldehyde (34.0 g, 1.08 mole), water (32.2 g, 1.79 mole), 2.5 g of 10% Pd/C, and DME (650 mL). A CO:H₂ (95:5) atmosphere at 1500 psi (10,345 kPa) was immediately established. This mixture was heated to 100° C. The reaction was warmed to 125° C. and maintained at this temperature for one hour. HPLC analysis showed a substantial yield of XVI.

### Example 11

This example demonstrates the regeneration and reuse a cobalt(II) salt from a carboxymethylation product mixture.

An autoclave was charged with cobalt acetate tetrahydrate (40.0 g, 0.158 mole), Co₂(CO)₈ (4.1 g, 0.012 mole) and acetic acid (102 g, 1.70 mole) and pressurized to 2200 psi (15,170 kPa) CO:H₂ (90:10) at 25° C. This mixture was heated to 130° C. for one hour. Gas uptake indicated that the cobalt(II) salt was converted to catalyst precursor.

The regenerated catalyst precursor was transferred under CO:H₂ pressure, into the autoclave at 95° C. containing CO:H₂ (95:5) at 900 psi (6,210 kPa), acetamide (VII)(59.0 g, 1.0 mole), 95% paraformaldehyde (68.0 g, 2,16 mole), water (64.5 g, 3.60 mole), 5.0 g. of 10% Pd/C, and DME (750 mL). A CO:H₂ atmosphere (95:5) at 1500 psi (10,345 kPa) was immediately established. This mixture was heated to 125° C. and maintained at this temperature for one hour. HPLC analysis of this stream indicated a substantial yield of XVI.

### Example 12

This example demonstrates the regeneration and reuse of a cobalt(II) salt to give high yields of XVI.

An autoclave was charged with cobalt acetate tetrahydrate (40.0 g, 0.158 mole), Co₂(CO)₈ (4.1 g, 0.012 mole) and acetic acid (100 g, 1.69 mole) and pressurized to 2200 psi (15,170 kPa) CO:H₂ (90:10) at 25° C. This mixture was heated to 130° C. for one hour. Gas uptake indicated that the cobalt(II) salt was converted to catalyst precursor.

The regenerated catalyst precursor was transferred under CO:H₂ pressure, into the autoclave at 95° C. containing CO:H₂ (95:5) at 900 psi (6,210 kPa), acetamide (VII)(59.0 g, 1.0 mole), 95% paraformaldehyde (68.0 g, 2,16 mole), water (64.5 g, 3.60 mole), 5.0 g of 10% Pd/C, and DME (600 mL). A CO:H₂ atmosphere (95:5) at 2200 psi (15,170 kPa) was immediately established. This mixture was heated to 125° C. and maintained at this temperature for one hour. HPLC analysis of this stream indicated a high yield of XVI.

### Example 13

This example demonstrates the regeneration of a cobalt(II) salt to an active carboxymethylation catalyst precursor in the presence of an amide after removal of a supported noble metal promoter such as 10% Pd/C from the carboxymethylation product mixture.

A 2 L autoclave was charged with acetamide (VII)(128.5 g, 2.2 mole), Co(OAc)₂.4H₂O (33 g, 0.13 mole), THF (750 mL), and acetic acid (250 mL). After sealing the autoclave, 2200 psi (15,172 kPa) of CO:H₂ (70:30) was established at 25° C. with stirring at 2000 rpm. The contents of the autoclave were heated to 130° C. at 3200 psi (22,069 kPa) CO:H₂ (70:30). After approximately 10 min., rapid gas uptake was observed indicative of regeneration to an active carboxymethylation catalyst.

### Example 14

The following examples demonstrate the use of different amides in the process of the invention.

A) A 2 L autoclave was charged with urea (V)(60 g, 1.0 mole), Co(OAc)₂.4H₂O (66 g, 0.26 mole), 10% Pd/C (5.0 g), and acetic acid (1 L). After sealing the autoclave, 2200 psi (15,172 kPa) of CO:H₂ (70:30) was established at 25° C. with stirring at 2000 rpm. The contents of the autoclave were heated to 130° C. at 3200 psi (22,069 kPa) CO:H₂ (70:30). After approximately one hour, rapid gas uptake was observed. The reaction mass was cooled to 85° C. and the feed gas was changed to a CO:H₂ (90:10) composition. Under a constant 3200 psi (22,069 kPa), 47 Wt. % formalin (320 mL, 5.28 mole) was delivered at 16 mL/min. The reaction was stirred at 85° C. for 90 min. after the formalin addition was complete. Then the reaction was cooled to 25° C., removed from the autoclave, and concentrated under reduced pressure. The residue was treated with 2 L of 10% HCl at 100° C. for two hours to yield (XIV) and glycine.

B) A 2 L autoclave was charged with methylene bisacetamide (VI)(130 g, 1.0 mole), Co(OAc)₂.4H₂O (49 g, 0.20 mole), 5% Pd/C (10.0 g), and THF (1 L). After sealing the autoclave, 2200 psi (15,172 kPa) of CO:H₂ (70:30) was established at 25° C. with stirring at 2000 rpm. The contents of the autoclave were heated to 130° C. at 3200 psi (22,069 kPa) CO:H₂ (70:30). After approximately 0.5 h, rapid gas uptake was observed. Under a constant 3200 psi (22,069 kPa), 47 Wt. % formalin (300 mL, 4.95 mole) was delivered at 10 mL/min. The reaction was stirred at 130° C. for 60 min. after the formalin addition was complete. Then the reaction was cooled to 25° C., removed from the autoclave and assayed indicating a substantial yield of XVI.

C) A 1 L autoclave was charged with N-methyl acetamide (IX)(160 g, 2.2 mole), Co(OAc)₂.4H₂O (33 g, 0.13 mole), 10% Pd/C (11.0 g), and acetic acid (1 L). After sealing the autoclave, 2200 psi (15,172 kPa) CO:H₂ (70:30) was established at 25° C. with stirring at 2000 rpm. The contents of the autoclave were heated to 130° C., and 3200 psi (22,069 kPa) CO:H₂ (70:30) was established. After approximately 0.5 h, rapid gas uptake was observed. The reaction mass was cooled to 85° C. Under a constant 3200 psi (22,069 kPa), 47 Wt. % formalin (180 mL, 2.97 mole) was delivered at 6 mL/min. The reaction was stirred at 85° C. for 30 min. after the formalin addition was complete. The reaction was then cooled to 25° C., removed from the autoclave, and assayed for N-acetyl sarcosine confirming a high yield.

D) A 2 L autoclave was charged with 1,3-dimethylurea (X) (96.9 g, 1.1 mole), Co(OAc)₂.4H₂O (33 g, 0.13 mole), 10% Pd/C (5.5 g), and acetic acid (500 mL). After sealing the autoclave, 2200 psi (15,172 kPa) CO:H₂ (70:30) was established at 25° C. with stirring at 2000 rpm. The contents of the autoclave were heated to 130° C. and 3200 psi (22,069 kPa) CO:H₂ (70:30) was established. After approximately 1 h, rapid gas uptake was observed. The reaction mass was cooled to 85° C. Under a constant 3200 psi (22,069 kPa), 47 Wt. % formalin (201 mL, 3.31 mole) was delivered at 6 mL/min. The reaction was stirred at 85° C. for 60 min. after the formalin addition was complete. Then the reaction was cooled to 2° C., removed from the autoclave, and concentrated to an oil under reduced pressure. The oil was treated with 2 L of 10% HCl at 100° C. for two hours to afford a yield of XXIII.

E) A 1 L autoclave was charged with N,N′-bis-phosphonomethylurea (Xl)(12.3 g, 0.05 mole), Co(OAc)₂.4H₂O (2.4 g, 0.01 mole), 1.0% Pd/C (2.5 g), and acetic acid (300 mL). After sealing the autoclave, 2200 psi (15,172 kPa) CO:H₂ (70:30) was established at 25° C. with stirring at 2000 rpm. The contents of the autoclave were heated to 130° C., and 3200 psi (22,069 kPa) CO:H₂ (70:30) was established. After approximately 1.5 h, rapid gas uptake was observed. The reaction mass was cooled to 95° C. Under a constant 3200 psi (22,069 kPa), 47 Wt. % formalin (10 mL, 0.17 mole) was delivered at 0.5 mL/min. The reaction was stirred at 95° C. for 60 min. after the formalin addition was complete. Then the reaction was cooled to 25° C., removed from the autoclave, and concentrated to an oil under reduced pressure. The oil was treated with 500 mL of 10% HCl at 100° C. for two hours to afford N-phosphonomethyl)glycine (1).

F) A 300 mL autoclave was charged with N-acetyl glycine (XVIII) (23.4 g, 0.20 mole), 95% paraformaldehyde (6.8 g, 0.22 mole), water (6.5 g, 0.36 mole), acetic acid (16.8 g, 0.28 mole), DME (90 mL), 10% Pd/C (1.0 g), and Co₂(CO)₈ (2.01 g, 0.006 mole) and pressurized to 1500 psi (10,345 kPa) CO:H₂ (95:5) at 25° C. This mixture was heated to 110° C. for 30 min. HPLC analysis of this stream showed a high yield of XVI with a small amount of XIV and some unreacted XVIII.

G) A 2 L autoclave was charged with 130 g of a solid with a composition of 85% methylene bisacetamide (VI)/10% [CH₃C(O)N(H)CH₂]₂NC(O)CH₃/5% acetamide (VII), Co(OAc)₂.4H₂O (49 g, 0.20 mole), 10% Pd/C (5.0 g), and THF (1 L). After sealing the autoclave, 2200 psi (15,172 kPa) of CO:H₂ (70:30) was established at 25° C. with stirring at 2000 rpm. The contents of the autoclave were heated to 130° C. and 3200 psi (22,069 kPa) CO:H₂ (70:30) was established. After approximately 0.5 h, rapid gas uptake was observed. Under a constant 3200 psi (22,069 kPa), 47 Wt. % formalin (300 mL, 4.95 mole) was delivered at 10 mL/min. The reaction was stirred at 130° C. for 60 min. after the formalin addition was complete. Then the reaction was cooled to 25° C. and removed from the autoclave. HPLC analysis of the reaction indicated a good yield of N-acetyl iminodiacetic acid (XVI).

H) A 2 L autoclave was charged with acetamide (VII)(128.5 g, 2.2 mole), Co(OAc)₂.4H₂O (33 g, 0.13 mole), THF (960 mL), 10% Pd/C (10.0 g), and acetic acid (40 mL). After sealing the autoclave, 2200 psi (15,172 kPa) of CO:H₂ (70:30) was established at 25° C. with stirring at 2000 rpm. The contents of the autoclave were heated to 130° C., and 3200 psi (22,069 kPa) CO:H₂ (70:30) was established. After approximately 75 min., rapid gas uptake was observed. The contents of the autoclave were cooled to 85° C., and 3200 psi (22,069 kPa) was established with a CO:H₂ (90:10) feed. Under a constant 3200 psi (22,069 kPa) (90:10/CO:H₂ feed), 47 Wt. % formalin (320 mL, 5.28 mole) was delivered at 9 mL/min. The reaction was stirred at 85° C. for 60 min. after the formalin addition was complete. Then the reaction was cooled to 25° C., removed from the autoclave, and assayed for N-acetyl iminodiacetic acid showing a high yield of XVI with detectable XVIII.

I) A 300 mL autoclave was charged with N-methane sulfonamide (19.0 g, 0.20 mole), 95% paraformaldehyde (6.8 g, 0.22 mole), water (6.5 g, 0.36 mole), acetic acid (16.8 g, 0.28 mole), DME (90 mL), 10% Pd/C (1.0 g), and Co₂(CO)₈ (2.01 g, 0.006 mole) and pressurized to 1500 psi (10,345 kPa) CO:H₂ (95:5) at 25° C. This mixture was heated to 110° C. for 30 min. HPLC analysis of this stream showed a yield of N-methane sulfonyliminodiacetic acid and N-methane sulfonylglycine.

J) A 300 mL autoclave was charged with N-methane sulfonylglycine (30.6 g, 0.20 mole), 95% paraformaldehyde (6.8 g, 0.22 mole), water (6.5 g, 0.36 mole), acetic acid (16.8 g, 0.28 mole), DME (90 mL), 10% Pd/C (1.0 g), and Co₂(CO), (2.01 g, 0.006 mole) and pressurized to 1500 psi (10,345 kPa) CO:H₂ (95:5) at 25° C. This mixture was heated to 110° C. for 30 min. HPLC analysis of this stream showed a yield of N-methane sulfonyliminodiacetic acid with detectable unreacted -methane sulfonylglycine.

### Examples 15

This example demonstrates the use of acetonitrile as a solvent in the carboxymethylation reaction of the present invention.

A 300 mL autoclave was charged with acetamide (VII)(11.8 g, 0.2 mole), 95% paraformaldehyde (13.6 g, 0.43 mole), water (12.9 g, 0.72 mol), acetic acid (16.8 g, 0.28 mole), acetonitrile (90 mL), 10% Pd/C (1.0 g), and Co₂(CO)₈ (4.1 g, 0.012 mole) and pressurized to 3200 psi (22,069 kPa) CO:H₂ (95:5) at 25° C. This mixture was heated to 110° C. for 30 min to form a product mixture. HPLC analysis of the product mixture indicated a high yield of XVI.

### Example 16

This example demonstrates the use of acetone as a solvent in the carboxymethylation reaction of the present invention.

A 300 mL autoclave was charged with acetamide (VII)(11.8 g, 0.2 mole), 95% paraformaldehyde (13.6 g, 0.43 mole), water (12.9 g, 0.72 mole), acetic acid (2.1 g, 0.035 mole), acetone (90 mL), 10% Pd/C (1.0 g), and Co₂(CO)₈ (2.1 g, 0.006 mol) to form a carboxymethylation reaction mixture and pressurized to 3200 psi (22,069 kPa) CO:H₂ (95:5) at 25° C. The reaction mixture was heated to 110° C. for 30 min to form a carboxymethylation product mixture. HPLC analysis of the product mixture indicated a high yield of XVI.

### Example 17

This example demonstrates the recovery of a supported noble metal promoter 10% Pd/C and cobalt(II) bis-N-acetyl iminodiacetate from a typical carboxymethylation product mixture.

A) An autoclave was charged with acetamide (11.8 g, 0.2 mole), 95% paraformaldehyde (13.6 g, 0.43 mole), water (12.9 g, 0.72 mol), acetic acid (33.0 g, 0.55 mole), acetone (70 g), 10% Pd/C (1.0 g), and Co₂(CO)₈ (2.55 g, 0.007 mole). After sealing the autoclave, 150 psi (1034 kPa) of CO:H₂ (95:5) was established at 25° C. and the autoclave was slowly vented. Then, 2200 psi (15,172 kPa) CO:H₂ (95:5) was established at 25° C. with stirring at 2000 rpm. The contents of the autoclave were heated to 100° C. and 3200 psi (22,069 kPa) CO:H₂ (95:5) was established. The reaction mixture was heated to 100° C. for 30 min. The carboxymethylation product mixture comprising the resulting reaction mass was allowed to settle and then decanted from the 10% Pd/C on the bottom of the vessel. The 10% Pd/C was then washed from the vessel by slurrying with acetone. Filtering the slurry gives 10% Pd/C that can be reused.

Of the decanted product mixture, 141.3 g were transferred to a round bottom flask. Air was bubbled through the stirred product mixture at room temperature for 130 min. until the solution turned dark purple with a slight cloudiness. The air supply was then turned off and the reaction mixture was heated at reflux for 80 min. Pink precipitate started to appear after 30 min. of heating and continued through the heating period. The system was cooled to 30° C. and the pink solid was filtered, washed with acetone, and dried to give a solid. This solid was the cobalt salt of N-acetyl iminodiacetic acid. Analysis of the liquid filtrate showed only traces of cobalt and a good yield of N-acetyl iminodiacetic acid.

B) An autoclave was charged with acetamide (11.8 g, 0.2 mole), 95% paraformaldehyde (13.6 g, 0.43 mole), water (12.9 g, 0.72 mol), acetic acid (33.0 g, 0.55 mol), acetone (70.1 g), 5% Pd/C (2.0 g), and Co₂(CO), (3.03 g, 0.009 mole). After sealing the autoclave, 150 psi (1034 kPa) CO:H₂ (95:5) was established at 25° C. and the autoclave was slowly vented. Then, 2200 psi (15,172 kPa) of CO:H₂ (95:5) was established at 25° C. with stirring at 2000 rpm. The contents of the autoclave were heated to 100° C., and 3200 psi (22,069 kPa) CO:H₂ (95:5) was established. The reaction mixture was heated to 100° C. for 30 min. The resulting carboxymethylation product mixture was then filtered under a pressure of CO:H₂ (95:5) at 100IC to separate and collect the 5% Pd/C. The 5% Pd/C was then washed with acetone and was available for reuse.

The product mixture (143.9 g) was transferred to a round bottom flask. Air was bubbled through the stirred product mixture while heating to 61.5° C. for 120 min. while air bubbling was continued. A pink precipitate first appeared after about 60 min. The system was cooled to 30° C., and the pink solid was filtered off, washed with acetone, and dried to give a solid consisting substantially of the cobalt salt of N-acetyl iminodiacetic acid. Analysis of the liquid filtrate showed only trace residues of cobalt with a high yield of N-acetyl iminodiacetic acid.

C) In a typical carboxymethylation reaction, a 300 mL autoclave was charged with a mixture of water (12.9 g), glacial acetic acid (33.0 g), tetrahydrofuran (90 mL), paraformaldehyde (13.6 g of 95+% powder), acetamide (11.8 g), 10% Pd/C (1.0 g), and Co₂(CO)₈ (2.078 g, equivalent to ca. 716 mg cobalt). A gas mixture of CO:H₂ (95:5) was charged at an initial pressure of 3200 psi (22,069 kPa), the reactor heated to 110° C. for 30 min. with stirring, and then cooled to below 20° C. The pressure was slowly vented, the system purged with N₂, and the reactor was opened under an inert atmosphere and its contents filtered into a 250 mL glass three-necked round bottom flask fitted with a gas inlet tube, a thermocouple thermometer, and a distillation head. The 10% Pd/C collected on the filter can be recycled. The vessel was heated under a N₂ atmosphere and the contents distilled (pot temp. −70-80° C., still head temp-64° C.) until a 60 mL distillate was collected. Pink precipitate formed in the bottoms during the distillation. After cooling, the bottoms were filtered to obtain a pink powder containing most of the cobalt used.

In view of the above, it will be seen that the several objects of the invention are achieved and other advantageous results attained. As various changes could be made in the above methods without departing from the scope of the invention, it is intended that all matter contained in the above description or shown in the accompanying schemes shall be interpreted as illustrative and not in a limiting sense.

What is claimed is:

1. A process for the preparation of an amino carboxylic acid or a derivative thereof by carboxymethylation of an amide, an amide precursor or an amide source compound, the process comprising:
introducing a carboxymethylation catalyst precursor, a promoter for conversion of the carboxymethylation catalyst precursor to an active catalyst species, an aldehyde or an aldehyde source compound, carbon monoxide, hydrogen and the amide, amide precursor or amide source compound into a carboxymethylation reaction zone to form a carboxymethylation reaction mixture; and
heating the carboxymethylation reaction mixture under pressure to produce a carboxymethylation product mixture containing an amino carboxylic acid product, a catalyst precursor reaction product and the promoter.

2. A process as set forth in claim 1 wherein said amide, amide precursor or amide source compound comprises a compound having the formula: 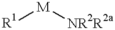
wherein;
M is selected from the group consisting of C(O), S(O), S(O)₂, P(O)OH, and P(O)R¹⁸;
R¹ is selected from the group consisting of hydrogen, hydrocarbyl, substituted hydrocarbyl, —NR³R⁴, —OR⁵, and —SR⁶;
R² and R^{2a} are independently selected from the group consisting of hydrogen, hydrocarbyl, and substituted hydrocarbyl;
R³ and R⁴ are independently selected from the group consisting of hydrogen, hydrocarbyl, and substituted hydrocarbyl;
R⁵ and R⁶ are independently selected from the group consisting of hydrogen, hydrocarbyl, substituted hydrocarbyl, and a salt-forming cation;
M and NR²R^{2a} are taken together to form a C≡N provided that R¹ is selected from the group consisting of hydrocarbyl and substituted hydrocarbyl; and
R¹⁸ is NR²R^{2a};
provided, however, that at least one of R² and R^{2a} is a group which undergoes carbonylation under the conditions of said carboxymethylation reaction; or R¹ is —NR³R⁴ and at least one of R³ and R⁴ is a group which undergoes carbonylation under the conditions of said carboxymethylation reaction.

3. A process as set forth in claim 2 wherein said carboxymethylation catalyst precursor comprises cobalt and the promoter comprises a metallic promoter.

4. A process as set forth in claim 3 wherein the metallic promoter is a supported noble metal promoter.

5. A process as set forth in claim 2 wherein said amide compound is a carbamoyl compound and said amino carboxylic acid product in said carboxymethylation product mixture comprises an N-acyl amino carboxylic acid or a derivative thereof,
said carbamoyl compound having the formula: 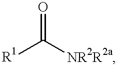
wherein;
R¹ is selected from the group consisting of hydrogen, hydrocarbyl, substituted hydrocarbyl, NR³R⁴, OR⁵, and SR⁶;
R² and R^{2a} are independently selected from the group consisting of hydrogen, hydrocarbyl, and substituted hydrocarbyl;
R³ and R⁴ are independently selected from the group consisting of hydrogen, hydrocarbyl, and substituted hydrocarbyl; and
R⁵ and R⁶ are independently selected from the group consisting of hydrogen, hydrocarbyl, substituted hydrocarbyl, and a salt-forming cation;
provided, however, that at least one of R² and R^{2a} is a group which undergoes carbonylation under the conditions of the carboxymethylation reaction; or R¹ is —NR³R⁴ and at least one of R³ and R⁴ is a group which undergoes carbonylation under the conditions of carboxymethylation reaction.

6. A process as set forth in claim 5 wherein the carboxymethylation catalyst precursor comprises cobalt and the promoter comprises a metallic promoter.

7. A process as set forth in claim 6 wherein the metallic promoter is a supported noble metal promoter.

8. A process as set forth in claim 7 wherein R¹ is alkyl; R² is selected from the group consisting of hydrogen, alkyl, hydroxymethyl, amidomethyl, phosphonomethyl, carboxymethyl, an ester of phosphonomethyl, a salt of phosphonomethyl, an ester of carboxymethyl and a salt of carboxymethyl; and R^{2a} is a group which undergoes carbonylation under the conditions of the carboxymethylation reaction.

9. A process as set forth in claim 8 wherein the N-acyl amino carboxylic acid product in said carboxymethylation product mixture is an N-alkyl-N-acyl amino carboxylic acid and the process further comprises oxidatively dealkylating said N-alkyl-N-acyl amino carboxylic acid by contacting said N-alkyl-N-acyl amino carboxylic acid with a noble metal catalyst in the presence of oxygen to produce N-(phosphonomethyl)glycine, a salt of N-(phosphonomethyl)glycine or an ester of N-(phosphonomethyl)glycine.

10. A process as set forth in claim 8 wherein the N-acyl amino carboxylic acid product in said carboxymethylation product mixture is an N-carboxymethyl-N-acyl amino carboxylic acid or an N-phosphonomethyl-N-acyl amino carboxylic acid and the process further comprises converting said N-carboxymethyl-N-acyl amino carboxylic acid or said N-phosphonomethyl-N-acyl amino carboxylic acid to N-(phosphonomethyl)glycine, a salt of N-(phosphonomethyl)glycine or an ester of N-(phosphonomethyl)glycine.

11. A process as set forth in claim 8 wherein R¹ is methyl or ethyl; R² is selected from the group consisting of hydrogen, methyl, isopropyl, hydroxymethyl, carboxymethyl, phosphonomethyl, an ester of phosphonomethyl, a salt of phosphonomethyl, an ester of carboxymethyl and a salt of carboxymethyl; and R^{2a} is selected from the group consisting of hydrogen and hydroxymethyl.

12. A process as set forth in claim 9 wherein said N-acyl amino carboxylic acid product is N-acetyl iminodiacetic acid.

13. A process as set forth in claim 12 further comprising converting said N-acetyl iminodiacetic acid product to iminodiacetic acid or a salt thereof.

14. A process as set forth in claim 9 wherein the molar ratio of noble metal content of the supported noble metal promoter to carbamoyl compound in the carboxymethylation reaction mixture is from about 0.0001 and about 0.05.

15. A process as set forth in claim 14 wherein the supported noble metal promoter comprises palladium or platinum.

16. A process as set forth in claim 15 wherein the supported noble metal promoter is palladium on a carbon support.

17. A process as set forth in claim 14 wherein the carboxymethylation reaction is conducted at a pressure of from about 700 kPa to about 28,000 kPa.

18. A process as set forth in claim 17 wherein the carboxymethylation reaction is conducted at a pressure of from about 3,500 kPa to about 24,500 kPa.

19. A process as set forth in claim 17 wherein the carboxymethylation reaction is conducted at a pressure of from about 4,200 kPa to about 22,750 kPa.

20. A process as set forth in claim 14 wherein the molar ratio of carbon monoxide to hydrogen in said carboxymethylation reaction zone is at least about 1:1.

21. A process as set forth in claim 20 wherein the molar ratio of carbon monoxide to hydrogen in said carboxymethylation reaction zone is from about 2:1 to about 99:1.

22. A process as set forth in claim 20 wherein the molar ratio of carbon monoxide to hydrogen in said carboxymethylation reaction zone is from about 6:1 to about 32:1.

23. A process as set forth in claim 14 wherein the molar ratio of cobalt atoms in said carboxymethylation catalyst precursor to carbamoyl compound in said carboxymethylation reaction zone is about 0.001 to about 1.

24. A process as set forth in claim 23 wherein the molar ratio of cobalt atoms in said carboxymethylation catalyst precursor to carbamoyl compound in said carboxymethylation reaction zone is about 0.01 to about 0.5.

25. A process as set forth in claim 23 wherein the molar ratio of cobalt atoms in said carboxymethylation catalyst precursor to carbamoyl compound in said carboxymethylation reaction zone is about 0.02 to about 0.1.

26. A process as set forth in claim 14 wherein water is introduced into said carboxymethylation reaction zone, the molar ratio of water to carbamoyl compound in said carboxymethylation reaction zone being less than about 10:1

27. A process as set forth in claim 26 wherein water is introduced into said carboxymethylation reaction zone, the molar ratio of water to carbamoyl compound in said carboxymethylation reaction zone being from about 2:1 to about 5:1.

28. A process as set forth in claim 26 wherein water is introduced into said carboxymethylation reaction zone, the molar ratio of water to carbamoyl compound in said carboxymethylation reaction zone being from about 3:1 to about 4:1.

29. A process as set forth in claim 14 wherein the carboxymethylation reaction mixture is heated to a temperature of from about 50° C. to about 170° C.

30. A process as set forth in claim 29 wherein the carboxymethylation reaction mixture is heated to a temperature of from about 65° C. to about 140° C.

31. A process as set forth in claim 29 wherein the carboxymethylation reaction mixture is heated to a temperature of from about 95° C. to about 115° C.

32. A process as set forth in claim 14 wherein said carboxymethylation reaction mixture further contains a solvent selected from the group consisting of an ether, a ketone, an ester, a nitrile, and mixtures thereof.

33. A process as set forth in claim 14 wherein said carboxymethylation reaction mixture further contains an acid having a pKa of less than about 3 selected from the group consisting of sulfuric acid, methanesulfonic acid, toluenesulfonic acid, trifluoromethanesulfonic acid, hydrochloric acid, phosphoric acid, triflouroacetic acid, and benzenephosphonic acid.

34. A process as set forth in claim 14 wherein said carboxymethylation reaction mixture further contains an acid having a pKa of greater than about 3 selected from the group consisting of acetic acid and propanoic acid is introduced into said carboxymethylation reaction zone.

35. A process as set forth in claim 14 wherein said aldehyde or aldehyde source compound is introduced into said carboxymethylation reaction zone in a molecular form, in aqueous solution, as an adduct of the amide, or as an acetal.

36. A process as set forth in claim 14 wherein said aldehyde or aldehyde source compound is formaldehyde, acetaldehyde, 3-methylthiopropionaldehyde or isobutyraldehyde.

37. A process as set forth in claim 14 wherein said aldehyde is formaldehyde and the source of said formaldehyde is formalin.

38. A process as set forth in claim 14 wherein the process further comprises recovering the promoter from said carboxymethylation product mixture;
recovering the catalyst precursor reaction product from said carboxymethylation product mixture; and
regenerating the carboxymethylation catalyst precursor from said recovered catalyst precursor reaction product in the presence of said amide compound.

39. A process as set forth in claim 38 wherein the catalyst precursor reaction product is recovered by exposing the carboxymethylation product mixture to a molecular oxygen containing gas, forming a solid containing a cobalt(II) salt in said product mixture, and filtering the solid from the product mixture.

40. A process as set forth in claim 39 wherein the formation of the solid is accelerated by the addition of an organic acid to the carboxymethylation product mixture, adding excess solvent to the carboxymethylation product mixture, or distilling solvent from the carboxymethylation product mixture.

41. A process as set forth in claim 38 wherein the catalyst precursor reaction product is recovered by forming a solid containing a cobalt(II) salt under anaerobic conditions, and filtering the solid from the carboxymethylation product mixture.

42. A process as set forth in claim 41 wherein the formation of the solid is accelerated by the addition of an organic acid to the carboxymethylation product mixture, adding excess solvent to the carboxymethylation product mixture, or distilling solvent from the carboxymethylation product mixture.

43. A process as set forth in claim 38 wherein the carboxymethylation catalyst precursor is regenerated in the presence of the carbamoyl compound.

44. A process as set forth in claim 38 wherein the cobalt(II) salt is regenerated using carbon monoxide and hydrogen to produce hydridocobalttetracarbonyl, said hydridocobalttetracarbonyl being recycled as a carboxymethylation catalyst precursor in additional carboxymethylation reactions.

45. A process as set forth in claim 6 wherein water and an aldehyde or an aldehyde source compound are introduced into the carboxymethylation reaction zone after the carbamoyl compound, the cobalt carboxymethylation catalyst precursor and the metallic promoter are contacted within said carboxymethylation reaction zone.

46. A process as set forth in claim 47 wherein R¹ is NR³R⁴; R² and R³ are independently selected from the group consisting of hydrogen, alkyl, hydroxymethyl, amidomethyl, phosphonomethyl, carboxymethyl, an ester of phosphonomethyl, a salt of phosphonomethyl, an ester of carboxymethyl, and a salt of carboxymethyl; and R^{2a} and R⁴ are independently selected from a group which undergoes carbonylation under the conditions of the present carboxymethylation process.

47. A process as set forth in claim 46 wherein R¹ is NR³R⁴; R² and R³ are independently selected from the group consisting of hydrogen, methyl, isopropyl, hydroxymethyl, carboxymethyl, phosphonomethyl, and an ester or salt of carboxymethyl or phosphonomethyl; and R^{2a} and R⁴ are independently selected from the group consisting of hydrogen and hydroxymethyl, and the metallic promoter is a supported noble metal promoter.

48. A process as set forth in claim 47 wherein the carbamoyl compound is selected from the group consisting of N-phosphonomethylurea, N,N′-bis-phosphonomethylurea, and urea.

49. A process as set forth in claim 48 wherein the N-acyl amino carboxylic acid product in said carboxymethylation product mixture is an N,N′-bis-phosphonomethylurea product or a urea product, and said process further comprises converting said N,N′-bis-phosphonomethylurea product or urea product to N-(phosphonomethyl)glycine or an ester or salt thereof.

50. A process as set forth in claim 48 wherein the N-acyl amino carboxylic acid product in said carboxymethylation product mixture is a urea product, and said process further comprises converting the urea product to iminodiacetic acid or an ester or salt thereof.

51. A process as set forth in claim 48 wherein the carboxymethylation reaction mixture further comprises an acid having a pKa of less than about 3 selected from the group consisting of sulfuric acid, methanesulfonic acid, toluenesulfonic acid, trifluoromethanesulfonic acid, hydrochloric acid, phosphoric acid, triflouroacetic acid, and benzenephosphonic acid.

52. A process as set forth in claim 48 wherein the carboxymethylation reaction mixture further comprises an acid having a pKa of greater than about 3 selected from the group consisting of acetic acid and propanoic acid.

53. A process as set forth in claim 45 wherein the process further comprises recovering the promoter from said carboxymethylation product mixture;
recovering the catalyst precursor reaction product from said carboxymethylation product mixture; and
regenerating the carboxymethylation catalyst precursor from said recovered catalyst precursor reaction product in the presence of said amide compound.

54. A process for the preparation of an amino carboxylic acid or a derivative thereof by carboxymethylation of an amide compound, the process comprising:
introducing a carboxymethylation catalyst precursor, a promoter for converting the carboxymethylation catalyst precursor to an active catalyst species, carbon monoxide, hydrogen, an aldehyde or an aldehyde source compound, and said amide compound into a carboxymethylation reaction zone to form a carboxymethylation reaction mixture;
heating the carboxymethylation reaction mixture under pressure to produce a carboxymethylation product mixture containing an N-acyl amino carboxylic acid product, a catalyst precursor reaction product, and the promoter;
recovering the promoter from said carboxymethylation product mixture;
recovering the catalyst precursor reaction product from said carboxymethylation product mixture; and
regenerating the carboxymethylation catalyst precursor from said recovered catalyst precursor reaction product in the presence of said amide compound,
said amide compound comprising a carbamoyl compound having the formula; 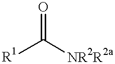
wherein
R¹ is selected from the group consisting of hydrocarbyl and substituted hydrocarbyl; and
R² and R^{2a} are independently selected from the group consisting of hydrogen, hydrocarbyl, and substituted hydrocarbyl;
provided, however, that at least one of R² and R^{2a} is a group which undergoes carbonylation under the conditions of the carboxymethylation reaction.

55. A process as set forth in claim 54 wherein the promoter is a metallic promoter and the carboxymethylation catalyst precursor comprises cobalt.

56. A process as set forth in claim 55 wherein the metallic promoter is a supported noble metal promoter.

57. A process as set forth in claim 54 wherein R¹ is alkyl; R² is selected from the group consisting of hydrogen, alkyl, hydroxymethyl, and amidomethyl; and R^{2a} is selected from the group consisting of hydrogen, hydroxymethyl, and amidomethyl.

58. A process as set forth in claim 57 wherein R¹ is C₈ to C₂₂ alkyl; R² is alkyl; and R^{2a} is selected from the group consisting of hydrogen and hydroxymethyl.

59. A process as set forth in claim 58 wherein the supported noble metal promoter comprises palladium or platinum.

60. A process as set forth in claim 59 wherein the carboxymethylation product mixture comprises an N—C₉ to C₂₃ alkanoyl sarcosine product or a salt thereof.

61. A process as set forth in claim 60 wherein the N—C₉ to C₂₃ alkanoyl sarcosine product is N-decanoylsarcosine, N-dodecanoylsarcosine, N-tetradecanoylsarcosine, and N-hexadecanoylsarcosine, or a salt thereof.

62. A process as set forth in claim 54 wherein the catalyst precursor reaction product is recovered by exposing the carboxymethylation product mixture to a molecular oxygen containing gas, forming a solid containing a cobalt(II) salt in said product mixture, and filtering the solid from the product mixture.

63. A process as set forth in claim 62 wherein the formation of the solid is accelerated by the addition of an organic acid to the carboxymethylation product mixture, adding excess solvent to the carboxymethylation product mixture, or distilling solvent from the carboxymethylation product mixture.

64. A process as set forth in claim 54 wherein the catalyst precursor reaction product is recovered by forming a solid containing a cobalt(II) salt under anaerobic conditions, and filtering the solid from the carboxymethylation product mixture.

65. A process as set forth in claim 64 wherein the formation of the solid is accelerated by the addition of an organic acid to the carboxymethylation product mixture, adding excess solvent to the carboxymethylation product mixture, or distilling solvent from the carboxymethylation product mixture.

66. A process as set forth in claim 54 wherein the carboxymethylation catalyst precursor is regenerated in the presence of the carbamoyl compound.

67. A process as set forth in claim 54 wherein the cobalt(II) salt is regenerated using carbon monoxide and hydrogen to produce hydridocobalttetracarbonyl, said hydridocobalttetracarbonyl being recycled as a carboxymethylation catalyst precursor in additional carboxymethylation reactions.

68. A process for the preparation of an amino carboxylic acid or a derivative thereof by carboxymethylation of an amide compound, the process comprising:
introducing a carboxymethylation catalyst precursor, a promoter for conversion of the carboxymethylation catalyst precursor to an active catalyst species, carbon monoxide, hydrogen, an aldehyde or an aldehyde source compound, and said amide compound into a carboxymethylation reaction zone to form a carboxymethylation reaction mixture;
heating the carboxymethylation reaction mixture under pressure to produce a carboxymethylation product mixture containing an amino carboxylic acid product, a catalyst precursor reaction product, and the promoter;
said amide compound comprising a sulfonamide or sulfinamide compound having the formula; 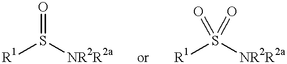
wherein;
R¹ is selected from the group consisting of hydrocarbyl, substituted hydrocarbyl, and NR³R⁴;
R² and R^{2a} are independently selected from the group consisting of hydrogen, hydrocarbyl, and substituted hydrocarbyl; and
R³ and R⁴ are independently selected from the group consisting of hydrogen, hydrocarbyl, and substituted hydrocarbyl;
provided, however, that at least one of R² and R^{2a} is a group which undergoes carbonylation under the conditions of the carboxymethylation reaction; or R¹ is —NR³R⁴ and at least one of R³ and R⁴ is a group which undergoes carbonylation under the conditions of the carboxymethylation reaction.

69. A process as set forth in claim 68 wherein the promoter is a metallic promoter and the carboxymethylation catalyst precursor comprises cobalt

70. A process as set forth in claim 69 wherein the metallic promoter is a supported noble metal promoter.

71. A process as set forth in claim 70 wherein R¹ is alkyl or aryl; R² is selected from the group consisting of hydrogen, alkyl, hydroxymethyl, amidomethyl, phosphonomethyl, carboxymethyl, an ester of the phosphonomethyl, a salt of the phosphonomethyl, an ester of the carboxymethyl, and a salt of the carboxymethyl; and R^{2a} is a group which undergoes carbonylation under the conditions of the present carboxymethylation process.

72. A process as set forth in claim 71 wherein R¹ is selected from the group consisting of methyl, ethyl, phenyl, and 4-methylphenyl; R² is selected from the group consisting of hydrogen, methyl, isopropyl, hydroxymethyl, carboxymethyl, phosphonomethyl, an ester of the phosphonomethyl, a salt of the phosphonomethyl, an ester of the carboxymethyl, and a salt of the carboxymethyl; and R^{2a} is selected from the group consisting of hydrogen and hydroxymethyl.

73. A process as set forth in claim 72 wherein the supported noble metal promoter comprises palladium or platinum.

74. A process as set forth in claim 68 wherein the process further comprises recovering the promoter from said carboxymethylation product mixture;
recovering the catalyst precursor reaction product from said carboxymethylation product mixture; and
regenerating the carboxymethylation catalyst precursor from said recovered catalyst precursor reaction product in the presence of said amide compound.

75. A process for the preparation of N-(phosphonomethyl)glycine or a salt or ester thereof, the process comprising:
preparing an N-acyl iminodiacetic acid by carboxymethylating an acylamide in a carboxymethylation reaction mixture formed by introducing said acylamide, water, formaldehyde, carbon monoxide, hydrogen, a supported noble metal promoter, and a carboxymethylation catalyst precursor comprising cobalt into a carboxymethylation reaction zone; and
deacylating said N-acyl iminodiacetic acid to convert said N-acyl iminodiacetic acid to N-(phosphonomethyl)glycine or a salt or ester thereof.

76. A process as set forth in claim 75 wherein the supported noble metal promoter comprises palladium or platinum, the acylamide is acetamide, and the N-acyl iminodiacetic acid is N-acetyl iminodiacetic acid.

77. A process as set forth in claim 76 wherein the supported noble metal promoter is palladium on carbon.

78. A process as set forth in claim 77 wherein the carboxymethylation reaction is conducted at a pressure of from about 700 kPa to about 28,000 kPa.

79. A process as set forth in claim 77 wherein the molar ratio of carbon monoxide to hydrogen in said carboxymethylation reaction zone is at least about 1:1.

80. A process as set forth in claim 77 wherein the molar ratio of cobalt atoms in said carboxymethylation catalyst precursor to acylamide in said carboxymethylation reaction zone is about 0.001 to about 1.

81. A process as set forth in claim 77 wherein water is introduced into said carboxymethylation reaction zone, the molar ratio of water to acylamide in said carboxymethylation reaction zone being less than about 10:1

82. A process as set forth in claim 77 wherein the carboxymethylation reaction mixture is heated to a temperature of from about 50° C. to about 170° C.

83. A process as set forth in claim 77 wherein said carboxymethylation reaction mixture further contains a solvent selected from the group consisting of an ether, a ketone, an ester, a nitrile, and mixtures thereof.

84. A process as set forth in claim 77 wherein said carboxymethylation reaction mixture further contains an acid having a pKa of less than about 3 selected from the group consisting of sulfuric acid, methanesulfonic acid, toluenesulfonic acid, trifluoromethanesulfonic acid, hydrochloric acid, phosphoric acid, triflouroacetic acid, and benzenephosphonic acid.

85. A process as set forth in claim 77 wherein said carboxymethylation reaction mixture further contains an acid having a pKa of greater than about 3 selected from the group consisting of acetic acid and propanoic acid is introduced into said carboxymethylation reaction zone.

86. A process as set forth in claim 85 wherein the carboxylic acid is acetic acid.

87. A process for the preparation of N-(phosphonomethyl)glycine, a salt of N-(phosphonomethyl)glycine, or an ester of N-(phosphonomethyl)glycine, the process comprising:
preparing an N-acyl amino carboxylic acid product by carboxymethylating a carbamoyl compound in a carboxymethylation reaction mixture formed by introducing said carbamoyl compound, formaldehyde, carbon monoxide, hydrogen, a carboxymethylation catalyst precursor comprising cobalt and a supported metallic promoter into a carboxymethylation reaction zone;
converting the N-acyl amino carboxylic acid product to N-(phosphonomethyl)glycine, a salt of N-(phosphonomethyl)glycine, or an ester of N-(phosphonomethyl)glycine wherein said conversion comprises deacylating the N-acyl amino carboxylic acid product to generate a carboxylic acid and an amino acid; and
reacting the carboxylic acid with an amine to generate the carbamoyl compound or a compound from which the carbamoyl compound may be derived;
said carbamoyl compound having the formula: 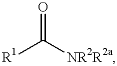
wherein;
R¹ is selected from the group consisting of hydrogen, hydrocarbyl, substituted hydrocarbyl, NR³R⁴, OR⁵, and SR⁶;
R² and R^{2a} are independently selected from the group consisting of hydrogen, hydrocarbyl, and substituted hydrocarbyl;
R³ and R⁴ are independently selected from the group consisting of hydrogen, hydrocarbyl, and substituted hydrocarbyl; and
R⁵ and R⁶ are independently selected from the group consisting of hydrogen, hydrocarbyl, substituted hydrocarbyl, and a salt-forming cation;
provided, however, that at least one of R² and R^{2a} is a group which undergoes carbonylation under the conditions of the carboxymethylation reaction; or R¹ is —NR³R⁴ and at least one of R³ and R⁴ is a group which undergoes carbonylation under the conditions of carboxymethylation reaction.

88. A process as set forth in claim 87 wherein the supported metallic promoter comprises a noble metal.

89. A process as set forth in claim 88 wherein the supported metallic promoter comprises palladium or platinum.

90. A process as set forth in claim 87 wherein the carbamoyl compound comprises an N-phosphonomethyl substituent.

91. A process as set forth in claim 87 wherein the carbamoyl compound is selected from the group consisting of acetamide and N-alkyl acetamide.

92. A process as set forth in claim 87 wherein the N-acyl amino carboxylic acid product contains an N-alkyl substituent, and the process further comprises
converting the N-acyl amino carboxylic acid product into an N-alkyl-N-(phosphonomethyl)glycine compound; and
oxidatively dealkylating the N-alkyl-N-(phosphonomethyl)glycine compound in the presence of oxygen using a noble metal catalyst.

93. A process as set forth in claim 87 wherein the process further comprises converting the N-acyl amino carboxylic acid product to a 2,5-diketopiperazine having the formula: 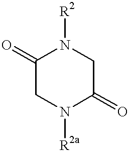
wherein R² and R^{2a} are hydrogen, alkyl, carboxymethyl, a salt of carboxymethyl, or an ester of carboxymethyl.

94. A process as set forth in claim 93 wherein the diketopiperazine is precipitated from the carboxymethylation product mixture.

95. A process as set forth in claim 87 wherein the carboxymethylation catalyst precursor is recovered by exposing the carboxymethylation product mixture to a molecular oxygen containing gas, forming a solid containing a cobalt(II) salt in said carboxymethylation product mixture, and filtering the solid from said product mixture.

96. A process as set forth in claim 95 wherein the formation of the solid is accelerated by the addition of an organic acid to the carboxymethylation product mixture, adding excess solvent to the carboxymethylation product mixture, or distilling solvent from the carboxymethylation product mixture.

97. A process as set forth in claim 87 wherein the carboxymethylation catalyst precursor is recovered by forming a solid containing a cobalt(II) salt in said carboxymethylation product mixture under anaerobic conditions, and filtering the solid from the carboxymethylation product mixture.

98. A process as set forth in claim 97 wherein the formation of the solid is accelerated by the addition of an organic acid to the carboxymethylation product mixture, adding excess solvent to the carboxymethylation product mixture, or distilling solvent from the carboxymethylation product mixture.

99. A process as set forth in claim 95 wherein the cobalt(II) salt is regenerated using carbon monoxide and hydrogen to produce hydridocobalttetracarbonyl, said hydridocobalttetracarbonyl being recycled as a carboxymethylation catalyst precursor in additional carboxymethylation reactions.

100. A process for the preparation of N-(phosphonomethyl)glycine, a salt of N-(phosphonomethyl)glycine, or an ester of N-(phosphonomethyl)glycine, the process comprising:
preparing N-acetyl iminodiacetic acid by carboxymethylating acetamide in a carboxymethylation reaction mixture formed by introducing acetamide, acetic acid, water, formaldehyde, carbon monoxide, hydrogen, a carboxymethylation catalyst precursor comprising cobalt and a supported noble metal promoter into a carboxymethylation reaction zone; and
converting the N-acetyl iminodiacetic acid to N-(phosphonomethyl)glycine, a salt of N-(phosphonomethyl)glycine, or an ester of N-(phosphonomethyl)glycine wherein said conversion comprises deacylating N-acetyl iminodiacetic acid.

101. A process as set forth in claim 100 wherein said supported noble metal promoter comprises palladium or platinum.

102. A process as set forth in claim 100 wherein the carboxymethylation reaction is conducted at a pressure of from about 700 kPa to about 28,000 kPa.

103. A process as set forth in claim 100 wherein the molar ratio of cobalt atoms in said carboxymethylation catalyst precursor to carbamoyl compound in said carboxymethylation reaction mixture is about 0.001 to about 1.

104. A process as set forth in claim 100 wherein the molar ratio of water to acetamide in the carboxymethylation reaction mixture is between about 2:1 and about 5:1.

105. A process as set forth in claim 100 wherein the process further comprises reacting the acetic acid with ammonia to produce acetamide.

106. A process as set forth in claim 100 wherein the process further comprises converting the N-acyl amino carboxylic acid reaction product to a 2,5-diketopiperazine having the formula 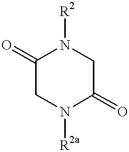
wherein R² and R^{2a} are carboxymethyl, a salt of carboxymethyl, or an ester of carboxymethyl.

107. A process as set forth in claim 105 wherein the diketopiperazine is precipitated from the carboxymethylation reaction mixture.

108. A process as set forth in claim 107 wherein a catalyst precursor reaction product is recovered from the carboxymethylation product mixture by refluxing the carboxymethylation product mixture under anaerobic conditions to form a solid containing a cobalt(II) salt in said product mixture, and filtering the solid from said product mixture.

109. A process as set forth in claim 107 wherein the catalyst precursor reaction product is recovered from the carboxymethylation reaction mixture by exposing the carboxymethylation product mixture to a molecular oxygen containing gas, forming a solid containing a cobalt(II) salt in said carboxymethylation product mixture, and filtering the solid from said product mixture.

110. A process as set forth in claim 109 wherein the formation of the solid is accelerated by the addition of an organic acid to the carboxymethylation product mixture, adding excess solvent to the carboxymethylation product mixture, or distilling solvent from the carboxymethylation product mixture.

111. A process as set forth in claim 107 wherein the catalyst precursor reaction product is recovered from the carboxymethylation product mixture by forming a solid containing a cobalt(II) salt in said product mixture under anaerobic conditions, and filtering the solid from said product mixture.

112. A process as set forth in claim 111 wherein the formation of the solid is accelerated by the addition of an organic acid to the carboxymethylation product mixture, adding excess solvent to the carboxymethylation product mixture, or distilling solvent from the carboxymethylation product mixture.

113. A process as set forth in claim 107 wherein the cobalt(II) salt is regenerated using carbon monoxide and hydrogen to produce hydridocobalttetracarbonyl, said hydridocobalttetracarbonyl being recycled as a carboxymethylation catalyst precursor.

114. A process as set forth in claim 113 wherein the cobalt(II) salt is regenerated in the presence of acetamide.

115. A process for the preparation of N-(phosphonomethyl)glycine, a salt of N-(phosphonomethyl)glycine, or an ester of N-(phosphonomethyl)glycine, the process comprising
introducing a carbamoyl compound, a carboxymethylation catalyst precursor, a supported noble metal promoter, formaldehyde, and carbon monoxide to a carboxymethylation reaction zone to form a carboxymethylation reaction mixture;
heating the carboxymethylation reaction mixture under pressure to produce a carboxymethylation product mixture containing an N-acyl amino carboxylic acid product and a catalyst precursor reaction product; and
converting the N-acyl amino carboxylic acid reaction product to N-(phosphonomethyl)glycine, a salt of N-(phosphonomethyl)glycine, or an ester of N-(phosphonomethyl)glycine,
said carbamoyl compound having the formula: 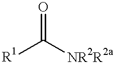
wherein;
R¹ is —NR³R⁴;
R² and R^{2a} are independently hydrogen, hydrocarbyl, or substituted hydrocarbyl; and
R³ and R⁴ are independently hydrogen, hydrocarbyl, or substituted hydrocarbyl;
provided, however, that at least one of R², R^{2a}, R³ and R⁴ is hydrogen, hydroxymethyl, amidomethyl, or another substituent which undergoes carbonylation under the conditions of the carboxymethylation reaction.

116. A process as set forth in claim 115 wherein said carboxymethylation catalyst precursor comprises cobalt and said supported noble metal promoter comprises palladium or platinum.

117. A process as set forth in claim 115 wherein the carbamoyl compound is urea, bisphonomethylurea, N-alkyl urea, or N,N′-dialkyl urea.

118. A process as set forth in claim 115 wherein the carbamoyl compound and the carboxymethylation catalyst precursor are introduced into the carboxymethylation reaction zone before introducing water and the formaldehyde into the carboxymethylation reaction zone.

119. A process for the preparation of N-(phosphonomethyl)glycine, a salt of N-(phosphonomethyl)glycine, or an ester of N-(phosphonomethyl)glycine, the process comprising
introducing a carbamoyl compound, a carboxymethylation catalyst precursor, a supported noble metal promoter, formaldehyde, and carbon monoxide into a carboxymethylation reaction zone to form a carboxymethylation reaction mixture;
reacting the components of the carboxymethylation reaction mixture to produce a carboxymethylation product mixture containing an N-acyl-N-alkyl amino carboxylic acid product and a catalyst precursor reaction product,
converting the N-acyl-N-alkyl amino carboxylic acid reaction product to an N-alkyl-N-(phosphonomethyl)glycine compound, and
oxidatively dealkylating the N-alkyl-N-(phosphonomethyl)glycine compound in the presence of oxygen using a noble metal catalyst,
said carbamoyl compound having the formula: 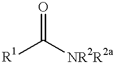
wherein;
R¹ is alkyl;
R² is hydrocarbyl or substituted hydrocarbyl; and
R^{2a} is hydrogen, hydroxymethyl, or another substituent which is carbonylated under the carboxymethylation reaction conditions.

120. A process as set forth in claim 119 wherein said carboxymethylation catalyst precursor comprises cobalt and said supported noble metal promoter comprises palladium or platinum.

121. A process as set forth in claim 119 wherein R² is methyl or isopropyl.

122. A process as set forth in claim 119 wherein the noble metal catalyst comprises platinum.
